(19) United States
(12) Patent Application Publication
(10) Pub. No.: US 2017/0188606 A1
(43) Pub. Date: Jul. 6, 2017

(54) COMPOUNDS, COMPOSITIONS, AND METHODS FOR REDUCING OR ELIMINATING BITTER TASTE

(71) Applicant: Chromocell Corporation, North Brunswick, NJ (US)

(72) Inventors: Amy Lancaster, Monmouth Junction, NJ (US); Leonard McDonald, Mountainside, NJ (US)

(21) Appl. No.: 15/308,711

(22) PCT Filed: May 7, 2015
(86) PCT No.: PCT/US15/29770

§ 371 Date: Nov. 3, 2016

Related U.S. Application Data
(60) Provisional application No. 61/989,878, filed on May 7, 2014.

(51) Int. Cl. A23L 2/56; C07H 7/02; A23L 27/30; A23L 27/00; A23L 2/60; A23L 27/21

(52) U.S. Cl. CPC A23L 2/56; A23L 2/60; A23L 27/21; A23L 27/36; A23L 27/86; C07H 7/02

(57) ABSTRACT

The present invention provides edible compositions comprising a compound of the present invention, food products comprising such edible compositions and methods of preparing such food products. The present invention also provides methods of reducing the amount of sugar in a food product, and methods of reducing bitter taste in a food product.

### RELATED APPLICATIONS

This application claims the benefit of U.S. provisional application 61/989,878, filed on May 7, 2014, which is incorporated by reference herein in its entirety.

### FIELD OF THE INVENTION

The present invention relates to flavor in edible compositions.

### BACKGROUND OF THE INVENTION

The sense of taste, e.g., in human, can detect at least five traditional tastes: sweet, sour, salty, bitter, and umami (savory). Many nutritious substances including vegetables, foods, food ingredients and nutrients comprise bitter tastants and/or have a bitter taste. In addition, many pharmaceutical substances important to maintain or improve health comprise bitter tastants and/or have a bitter taste. While certain food products and consumer products have desirable bitter tastes, including coffee, beer and dark chocolate, in many contexts, consumers dislike such bitter tastes. For example, many consumers dislike the perception of certain bitter tastants and/or bitter taste and will avoid food or pharmaceutical products with an undesirable bitter tastant or bitter taste in favor of food and pharmaceutical products that have reduced levels of undesirable biller tastants or that have reduced or completely lack bitter taste. This aversion to products containing undesirable bitter tastants and/or having undesirable bitter taste, by-taste or off-taste may be caused by perception of bitter tastants and/or bitter taste mediated by activation of bitter receptors present in the oral cavity and/or in the gastrointestinal tract. In many cases, consumer dislike of bitter tastants and/or bitter taste or off-taste, prevents or hampers improvement of the nutritive quality and safety of foods as desired levels of nutrients comprising bitter tastants and/or having bitter taste cannot be used. Also, dislike of or aversion to the bitter tastants or bitter taste of some pharmaceutical agents negatively impacts compliance with prescribed regimens for their use.

For instance, several additives, preservatives, emulsifiers and foodstuffs used in the production of ibod products comprise bitter tastants and/or have a bitter taste or off-taste. While these additives, preservatives, emulsifiers and foodstuffs may affect the taste of a food product, they may also be important for improving the nutritive quality of the food product.

For instance, the increasing incidence of obesity and diabetes has been attributed, in part, to the high sugar intake of many diets. Accordingly, substitution of sugar with another sweet tasting compound is desirable. Artificial and natural sugar substitutes that may be used to reduce sugar in foods are often associated with bitter taste, which again limits the extent to which these may be used to replace sugar in foods without causing adverse bitter taste. For example, a common sugar substitute is rebaudioside A, which also has a bitter taste in addition to its sweet taste.

Without being limited by theory, bitter, sweet, and umami tastants and compounds typically elicit a taste response via G-protein coupled receptors, while salty and sour tastants and compounds are typically hypothesized to elicit a taste response via ion channels. Bitter taste receptors belong to the T2R (also referred to as TAS2R) family of G-protein coupled receptors that induce intracellular calcium concentration changes in response to a bitter tastant. T2R receptors act via gustducin, a taste-specific G-protein. There are at least twenty-five different members of the T2R family, suggesting that the perception of bitter taste is complex, involving several different tastant-receptor interactions. Compounds capable of modulating the activation and/or signaling of bitter taste receptors in the oral cavity and/or the gastrointestinal tract could be effective to allow desired usage levels of bitter tastants or bitter tasting substances in food and pharmaceutical products without resulting in consumer dislike of such products due to perception of the increased levels of bitter tastants or bitter tastes. In some instances, blockers or modulators of bitter taste receptors and bitter taste may reduce the perception of bitter tastants and/or bitter taste via the bitter taste receptors and/or taste transduction signaling machinery present in the oral cavity and/or the gastrointestinal tract.

Traditionally, in food preparation and pharmaceuticals, bitter taste was masked using sweeteners and other tastants, including salt. In some cases, however, this is undesirable or insufficient because it can alter, mask, or interfere with other tastes/flavors/impressions (e.g., non-bitter tastes or desired bitter tastes) in the ibod product. Additionally, this approach has rarely been able to completely mask the bitter taste present in such food products or pharmaceuticals. For that reason, compounds which reduce bitter taste instead of, or in addition to, masking agents are preferred.

It is, therefore, desirable to provide compounds or preparations containing compounds that may be added to food products, consumer products and pharmaceuticals comprising bitter tastants or having a bitter taste to eliminate, modulate or reduce the perception of the bitter tastants or bitter taste. Similarly, it is desirable to provide food products, consumer products, and pharmaceutical compositions comprising such compounds. It is also desirable to decrease the sugar intake of a subject using such compounds to eliminate, modulate or reduce the perception of bitter taste associated with sugar substitutes.

### SUMMARY OF THE INVENTION

The present invention provides compounds that modulate bitter taste, edible compositions comprising such compounds, and methods of preparing such edible compositions. The present invention also provides methods of reducing the amount of sugar in an edible composition and methods of reducing bitter taste of an edible composition. The present invention further provides a method of reducing, modulating or eliminating the bitter taste of a food, consumer or pharmaceutical product in a subject. The present invention also provides a method of modulating, particularly reducing, the activation of a bitter taste receptor.

### Edible Compositions

One aspect of the present invention provides edible compositions for reducing bitter taste of a bitter tastant. In some embodiments, the edible composition comprises a modified amino acid compound. In some embodiments, the modified amino acid compound is a compound having a molecular weight less than about 1000, 500, or 300 daltons. In certain embodiments, the modified amino acid compound is Compound 1 or a comestibly or biologically acceptable salt or derivative thereof, or an enantiomer thereof.

In some embodiments, the edible composition for reducing bitter taste of a bitter tastant comprises a compound of Formula (I) is a comestibly or biologically acceptable salt or derivative thereof, or an enantiomer or diastereomer thereof. In some embodiments, the compound of Formula (I) has a molecular weight less than about 1000, 500, or 300 daltons. In certain embodiments, the compound of Formula (I) is Compound 1 or a comestibly or biologically acceptable salt or derivative thereof, or an enantiomer thereof.

In certain embodiments, the compound of Formula (I) is N-methyl-L-tryptophan or a comestibly or biologically acceptable salt or derivative thereof, or an enantiomer thereof.

In another embodiment, the edible composition comprises Compound 1, or a comestibly or biologically acceptable salt or derivative thereof, or an enantiomer thereof, as described herein, and a bitter tastant.

According to the invention, the bitter tastant can be inherent in, e.g., a food product (such as coffee or chocolate) or can be a component of an edible composition (such as a bitter tasting sweetener).

In another aspect of the invention, the edible composition is a food product comprising at least one compound of the invention. In certain embodiments, the compound of the invention is a compound of Formula (I), or a comestibly or biologically acceptable salt, derivative, enantiomer, or diastereomer thereof. In another embodiment, the compound of the invention is Compound 1 or a comestibly or biologically acceptable salt, derivative, or enantiomer thereof.

In another aspect of the present invention, the edible composition is a pharmaceutical composition comprising a bitter tasting pharmaceutically active ingredient and a compound of Formula (I), or a comestibly or biologically acceptable salt, derivative, enantiomer, or diastereomer thereof. In some embodiments, the pharmaceutical composition comprises a bitter tasting pharmaceutically active ingredient and Compound 1, or a comestibly or biologically acceptable salt, derivative, or enantiomer thereof.

In yet further embodiments, the edible composition is a pharmaceutical composition comprising a pharmaceutically active ingredient, a bitter tastant, and a compound of Formula (I), or a comestibly or biologically acceptable salt, derivative, enantiomer, or diastereomer thereof, as described herein, or combinations of any of the aforementioned. In yet further embodiments, the pharmaceutical composition comprises a pharmaceutically active ingredient, a bitter tastant, and Compound 1, or a comestibly or biologically acceptable salt, derivative, or enantiomer thereof, as described herein, or combinations of any of the aforementioned.

In another aspect of the present invention, the edible composition is a consumer product comprising a bitter tastant and a compound of Formula (I), or a comestibly or biologically acceptable salt derivative, enantiomer, or diastereomer thereof, as described herein, or combinations of any of the aforementioned. In some embodiments, the consumer product comprises a bitter tastant and Compound 1, or a comestibly or biologically acceptable salt, derivative, or enantiomer thereof, as described herein, or combinations of any of the aforementioned.

Yet another embodiment of the present invention provides a consumer product for reducing bitter taste of a bitter tastant, wherein said consumer product comprises a compound of Formula (1), or a comestibly or biologically acceptable salt, derivative, enantiomer, or diastereomer thereof, as described herein, or combinations of any of the aforementioned. In yet further embodiments, the consumer product for reducing bitter taste of a bitter tastant comprises Compound 1, or a comestibly or biologically acceptable salt, derivative, or enantiomer thereof, as described herein, or combinations of any of the aforementioned.

In another aspect of the present invention the edible composition is an intermediate product for the manufacturing of one or more food products, consumer products or pharmaceutical compositions comprising a bitter tastant and a compound of Formula (I), or a comestibly or biologically acceptable salt, derivative, enantiomer, or diastereomer thereof, as described herein, or combinations of any of the aforementioned. In some embodiments, the intermediate product comprises a bitter tastant and Compound 1, or a comestibly or biologically acceptable salt, derivative, or enantiomer thereof, as described herein, or combinations of any of the aforementioned.

In a further aspect, the present invention provides a method of preparing an edible composition comprising:
(a) providing a comestibly acceptable carrier, and
(b) adding to the comestibly acceptable carrier a compound of Formula (I), or a comestibly or biologically acceptable salt, derivative, enantiomer, or diastereomer thereof as described herein, or combinations of any of the aforementioned.

In another embodiment, the method of preparing an edible composition comprises:
(a) providing a comestibly acceptable carrier, and
(b) adding to the comestibly acceptable carrier Compound 1, or a comestibly or biologically acceptable salt, derivative, or enantiomer thereof, as described herein, or combinations of any of the aforementioned.

In some embodiments, the edible composition is a ibod product, a consumer product or a pharmaceutical composition. In some embodiments, the comestibly acceptable carrier is a foodstuff, a food product, or a pharmaceutically acceptable carrier.

In some embodiments, the comestibly acceptable carrier is inherently bitter. In such embodiments, the comestibly acceptable carrier may inherently contain a bitter tastant (i.e., the comestibly acceptable carrier is bitter without addition of a bitter tastant).

In some embodiments, the method of preparing an edible composition further comprises: (c) adding a bitter tastant.

In another embodiment, the invention provides a method of reducing the amount of sugar in an edible composition comprising:
(a) replacing an amount of sugar used in preparing an edible composition with an amount of a high potency sweetener, and
(b) incorporating into the edible composition an effective amount of a compound of Formula (I), or a comestibly or biologically acceptable salt, derivative, enantiomer, or diastereomer thereof, as described herein, or combinations of any of the aforementioned.

In another embodiment, the invention provides a method of reducing the amount of sugar in an edible composition comprising:
(a) replacing an amount of sugar used in preparing an edible composition with an amount of a high potency sweetener, and
(b) incorporating into the edible composition an effective amount of Compound 1, or a comestibly or biologically acceptable salt, derivative, or enantiomer thereof, as described herein, or combinations of any of the aforementioned.

In some embodiments, the edible composition is a food product, a consumer product or a pharmaceutical composition.

In some embodiments of the present invention, the method of reducing the amount of sugar in an edible composition comprises incorporating into the edible composition an amount of the compound sufficient to permit replacement of up to 25% of the sugar present in an edible composition with a high potency sweetener (e.g., rebaudioside A or stevioside). In some embodiments, the amount of the compound incorporated into the edible composition is sufficient to permit replacement of up to 50% of the sugar present in an edible composition with a high potency sweetener (e.g., rebaudioside A or stevioside). In yet further embodiments, the amount of the compound incorporated into the edible composition is sufficient to permit replacement of up to 75% of the sugar present in an edible composition with a high potency sweetener (e.g., rebaudioside A or stevioside). In some embodiments, the amount of the compound incorporated into the edible composition is sufficient to permit replacement of up to 100% of the sugar present in an edible composition with a high potency sweetener (e.g., rebaudioside A or stevioside). In some embodiments, the edible composition maintains a sweet flavor.

The present invention also provides a method of reducing the bitter taste attributed to a bitter tastant in an edible composition comprising adding an effective amount of a compound of Formula (I), or a comestibly or biologically acceptable salt, derivative, enantiomer, or diastereomer thereof, as described herein, or combinations of any of the aforementioned, to the edible composition such that any bitter taste induced by the bitter tastant is reduced. In some embodiments, the compound added to the edible composition is Compound 1, or a comestibly or biologically acceptable salt, derivative, or enantiomer thereof, as described herein, or combinations of any of the aforementioned.

The present invention further provides a method of reducing the bitter taste attributed to a bitter tastant in an edible composition comprising ingesting an effective amount of a compound of Formula (I), or a comestibly or biologically acceptable salt, derivative, enantiomer, or diastereomer thereof, as described herein, or combinations of any of the aforementioned, before, along with, or after the edible composition such that any bitter taste induced by the bitter tastant is reduced. In some embodiments, the compound ingested with the edible composition is Compound 1, or a comestibly or biologically acceptable salt, derivative, or enantiomer thereof, as described herein, or combinations of any of the aforementioned.

In some embodiments, the edible composition is a food product, a consumer product or a pharmaceutical composition.

In some embodiments, the method reduces the bitter taste induced by the bitter tastant by up to 25%. In some embodiments, the method reduces the bitter taste induced by the bitter tastant by up to 50%. In some embodiments, the bitter taste induced by the bitter tastant is reduced by up to 75%. In yet some embodiments, the bitter taste induced by the bitter tastant is reduced by up to 100%.

The present invention also provides a method of reducing or eliminating bitter taste in a subject utilizing an edible composition comprising a compound of Formula (I), or a comestibly or biologically acceptable salt, derivative, enantiomer, or diastereomer thereof as described herein, or combinations of any of the aforementioned. In some embodiments, the composition that reduces or eliminates a bitter taste in a subject comprises Compound 1, or a comestibly or biologically acceptable salt, derivative, or enantiomer thereof, as described herein, or combinations of any of the aforementioned.

The present invention also provides a method of reducing or eliminating the bitter taste and the lingering sweetness of high potency sweeteners in a subject utilizing an edible compositions comprising a compound of Formula I, or a comestibly or biologically acceptable salt, derivative, enantiomer, or diastereomer thereof, as described herein, or combinations of any of the aforementioned. In some embodiments, the composition that reduces or eliminates bitter taste and lingering sweetness in a subject comprises Compound 1, or a comestibly or biologically acceptable salt, derivative, or enantiomer thereof, as described herein, or combinations of any of the aforementioned.

Particular embodiments of the invention are set forth in the following numbered embodiments:
1. A composition comprising a compound of Formula (I):

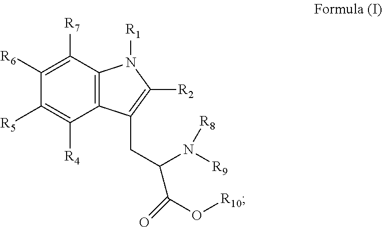

or a comestibly or biologically acceptable salt, derivative, diastereomer, or enantiomer thereof,
wherein, as valance and stability permit:
R₁ is independently H or C₁-C₆ alkyl;
R₂ is independently H or C₁-C₆ alkyl;
R₄ is independently H, OH, C₁-C₆ alkyl, or O(C₁-C₆) alkyl;
R₅ is independently H, OH, C₁-C₆ alkyl, or O(C₁-C₆) alkyl;
R₆ is independently H, OH, C₁-C₆ alkyl, or O(C₁-C₆) alkyl;
R₇ is independently H, OH, C₁-C₆ alkyl, or O(C₁-C₆) alkyl;
R₈ is independently H, C₁-C₆ alkyl, C(O)C₁-C₆ alkyl, C(O)R₁₁, or R₁₂;
R₉ is independently H, C₁-C₆ alkyl, C(O)C₁-C₆ alkyl, C(O)R₁₁, or R₁₂;
R₁₀ is independently H or C₁-C₆ alkyl;

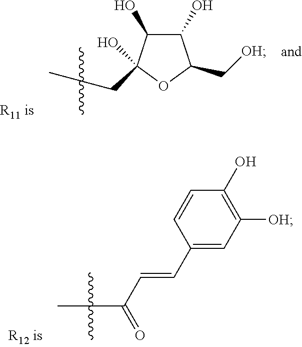 [SMILES: *C[C@]1(O)O[C@H](CO)[C@@H](O)[C@@H]1O ; *C(=O)/C=C/c1ccc(O)c(O)c1]

wherein the composition is edible and capable of reducing bitter taste of a bitter tastant.
2. The composition according to embodiment 1, or a comestibly or biologically acceptable salt, derivative, or enantiomer thereof, wherein as valence and stability permit:
R₁ is independently H or C₁-C₆ alkyl;
R₂ is independently H or C₁-C₆ alkyl;
R₄ is independently H, OH, C₁-C₆ alkyl, or O(C₁-C₆) alkyl;
R₅ is independently H, OH, C₁-C₆ alkyl, or O(C₁-C₆) alkyl;
R₆ is independently H, OH, C₁-C₆ alkyl, or O(C₁-C₆) alkyl;
R₇ is independently H, OH, C₁-C₆ alkyl, or O(C₁-C₆) alkyl;
R₈ is independently H, C₁-C₆ alkyl, or C(O)C₁-C₆ alkyl;
R₉ is independently H, C₁-C₆ alkyl, or C(O)C₁-C₆ alkyl; and
R₁₀ is independently H or C₁-C₆ alkyl.
3. The composition according to embodiment 1, or a comestibly or biologically acceptable salt, derivative, or enantiomer thereof, wherein as valence and stability permit:
R₁ is H;
R₂ is H;
R₄ is independently H, OH, or O(C₁-C₆) alkyl;
R₅ is independently H, OH, or O(C₁-C₆) alkyl;
R₆ is independently H, OH, or O(C₁-C₆) alkyl;
R₇ is independently H, OH, or O(C₁-C₆) alkyl;
R₈ is independently H or C₁-C₆ alkyl;
R₉ is independently H or C₁-C₆ alkyl; and
R₁₀ is independently H or C₁-C₆ alkyl.
4. The composition according to embodiment 1, or a comestibly or biologically acceptable salt, derivative, or enantiomer thereof, wherein as valence and stability permit
R₁ is H; R₂ is H; R₄ is H; R₅ is H; R₆ is H; R₇ is H; R₈ is H; R₉ is C₁-C₆ alkyl; and R₁₀ is H.
5. The composition according to embodiment 1, wherein said compound of Formula (I) is:

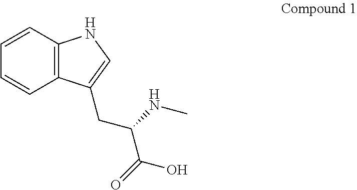 [SMILES: CN[C@@H](Cc1c[nH]c2ccccc12)C(=O)O]

or a comestibly or biologically acceptable salt, derivative, or enantiomer thereof.
6. The composition of any one of embodiments 1-5, wherein the composition further comprises a bitter tastant.
7. The composition according to embodiment 6, wherein the bitter tastant is a foodstuff.
8. The composition of according to embodiment 6, wherein the bitter tastant is a high potency sweetener.
9. The composition according to embodiment 8, wherein the high potency sweetener is stevioside or rebaudioside A.
10. The composition according to embodiment 9, wherein the high potency sweetener is rebaudioside A.
11. The composition of any one of embodiments 1-10, wherein the composition further comprises sugar.
12. A food product comprising the composition of any one of embodiments 1-11.
13. The food product of embodiment 12, wherein the food product is a beverage.
14. The food product of embodiment 12 or 13, wherein the food product further comprises a sweet taste improving composition.
15. A method of preparing an edible composition comprising:
(a) providing a comestibly acceptable carrier; and
(b) adding to the comestibly acceptable carrier a compound of Formula (I), or a comestibly or biologically acceptable salt, derivative, diastereomer, or enantiomer thereof; or Compound 1, or a comestibly or biologically acceptable salt, derivative, or enantiomer thereof; or combinations of any of the aforementioned.
16. The method according to embodiment 15, wherein said comestibly acceptable carrier is inherently bitter.
17. The method according to embodiment 15 or 16, wherein the edible composition further comprises sugar.
18. The method according to embodiment 17, wherein the method further comprises:
(c) adding a bitter tastant.
19. The method according to embodiment 18, wherein the bitter tastant comprises a high potency sweetener.
20. The method according to embodiment 19, wherein the high potency sweetener is stevioside or rebaudioside A.
21. The method according to embodiment 20, wherein the high potency sweetener is rebaudioside A.
22. A method of reducing the amount of sugar in an edible composition comprising:
   (a) replacing an amount of sugar present an edible composition with an amount of a high potency sweetener, and
   (b) incorporating into the edible composition an effective amount of a compound of Formula (I), or a comestibly or biologically acceptable salt, derivative, diastereomer, or enantiomer thereof; Compound 1, or a comestibly or biologically acceptable salt, derivative, or enantiomer thereof; or combinations of any of the aforementioned.
23. The method according to embodiment 22, wherein the amount of compound added is sufficient to permit replacement of the amount of sugar typically present in the edible composition by up to 25%.
24. The method according to embodiment 22, wherein the amount of compound added is sufficient to permit replacement of the amount of sugar typically present in the edible composition by up to 50%.
25. The method according to embodiment 22, wherein the amount of compound added is sufficient to permit replacement of the amount of sugar typically present in the edible composition by up to 75%.
26. The method according to embodiment 22, wherein the amount of compound added is sufficient to permit replacement of the amount of sugar typically present in the edible composition by up to 100%.
27. The method according to any one of embodiments 22-26, wherein the edible composition maintains a sweet flavor.
28. The method according to embodiment 27, wherein the high potency sweetener is stevioside or rebaudioside A.
29. The method according to embodiment 28, wherein the high potency sweetener is rebaudioside A.
30. A method of reducing bitter taste attributed to a bitter tastant in an edible composition comprising:
   (a) adding an effective amount of a compound of Formula (I), or a comestibly or biologically acceptable salt, derivative, diastereomer, or enantiomer thereof; Compound 1, or a comestibly or biologically acceptable salt, derivative, or enantiomer thereof; or combinations of any of the aforementioned, to the edible composition such that any bitter taste induced by the bitter tastant is reduced.
31. A method of reducing bitter taste attributed to a bitter tastant in an edible composition comprising:
   (a) ingesting an effective amount of a compound of Formula (I), or a comestibly or biologically acceptable salt, derivative, diastereomer, or enantiomer thereof; Compound 1, or a comestibly or biologically acceptable salt, derivative, or enantiomer thereof; or combinations of any of the aforementioned, along with the edible composition such that any bitter taste induced by the bitter tastant is reduced.
32. The method according to embodiment 30 or 31, wherein the edible composition is a food product, a consumer product, or a pharmaceutical composition.
33. The method according to any one of embodiments 30-32, wherein the bitter taste induced by the bitter tastant is reduced by up to 25%
34. The method according to any one of embodiments 30-32, wherein the bitter taste induced by the bitter tastant is reduced by up to 50%
35. The method according to any one of embodiments 30-32, wherein the bitter taste induced by the bitter tastant is reduced by up to 75%
36. The method according to any one of embodiments 30-32, wherein the bitter taste induced by the bitter tastant is reduced by up to 100%
37. A method of inhibiting, reducing, or eliminating a bitter taste in a subject comprising:
(a) placing a compound of Formula (I), or a comestibly or biologically acceptable salt, derivative, diastereomer, or enantiomer thereof; Compound 1, or a comestibly or biologically acceptable salt, derivative, or enantiomer thereof; or combinations of any of the aforementioned, in the oral cavity of the subject.
38. A pharmaceutical composition comprising:
   (a) a bitter tasting pharmaceutical active ingredient; and
   (b) a compound of Formula (I), or a comestibly or biologically acceptable salt, derivative, diastereomer, or enantiomer thereof; Compound 1, or a comestibly or biologically acceptable salt, derivative, or enantiomer thereof; or combinations of any of the aforementioned.
39. A pharmaceutical composition comprising:
   (a) a pharmaceutical active ingredient:
   (b) a bitter tastant; and
   (c) a compound of Formula (I), or a comestibly or biologically acceptable salt, derivative, diastereomer, or enantiomer thereof; Compound 1, or a comestibly or biologically acceptable salt, derivative, or enantiomer thereof; or combinations of any of the aforementioned.
40. The pharmaceutical composition according to embodiment 39, wherein the bitter tastant comprises a high potency sweetener.
41. The pharmaceutical composition according to embodiment 40, wherein the high potency sweetener is stevioside or rebaudioside A.
42. The pharmaceutical composition according to embodiment 41, wherein the high potency sweetener is rebaudioside A.
43. The pharmaceutical composition according to any one of embodiments 39-42 further comprising a sweet taste improving composition.
44. A consumer product comprising:
   (a) a bitter tasting ingredient; and
   (b) a compound of Formula (I), or a comestibly or biologically acceptable salt, derivative, diastereomer, or enantiomer thereof; Compound 1, or a comestibly or biologically acceptable salt, derivative, or enantiomer thereof; or combinations of any of the aforementioned.
45. A consumer product for reducing the bitter taste of a bitter tastant, wherein said consumer product comprises:
   (a) a compound of Formula (I), or a comestibly or biologically acceptable salt, derivative, diastereomer, or enantiomer thereof; Compound 1, or a comestibly or biologically acceptable salt, derivative, or enantiomer thereof; or combinations of any of the aforementioned.
46. A method for improving the taste of edible compositions comprising one or more high potency sweeteners, wherein said method comprises (a) adding an effective amount of a compound of Formula (I), or a comestibly or biologically acceptable salt, derivative, diastereomer, or enantiomer thereof; Compound 1, or a comestibly or biologically acceptable salt, derivative, or enantiomer thereof; or combinations of any of the aforementioned.
47. The method according to embodiment 46, wherein the high potency sweetener is stevioside or rebaudioside A.
48. The method according to embodiment 47, wherein the high potency sweetener is rebaudioside A.
49. An edible composition comprising one or more high potency sweeteners and an effective amount of compound of Formula (I), or a comestibly or biologically acceptable salt, derivative, diastereomer, or enantiomer thereof; Compound 1, or a comestibly or biologically acceptable salt, derivative, or enantiomer thereof; or combinations of any of the aforementioned.
50. The edible composition of embodiment 49, wherein the high potency sweetener is stevioside or rebaudioside A.
51. The edible composition of embodiment 50, wherein the high potency sweetener is rebaudioside A.
52. The edible composition according to any one of embodiments 49-51 further comprising a sweet taste modifying composition.

### BRIEF DESCRIPTION OF THE DRAWINGS

FIG. 1: Evaluation of the effects of Compound 1 on rebaudioside A activation of a mammalian cell system expressing a bitter taste receptor (T2R). Inhibition of the rebaudioside A response was observed with addition of Compound 1, and the fluorescent response from Compound 1 on its own was not significantly different from vehicle control.

### DETAILED DESCRIPTION OF THE INVENTION

In order that the invention described herein may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Chemistry terms used herein are used according to conventional usage in the art, as exemplified by “The McGraw-Hill Dictionary of Chemical Terms”, Parker S., Ed., McGraw-Hill, San Francisco, C.A. (1985).

Throughout this specification, the word “comprise” or variations such as “comprises” or “comprising” will be understood to imply the inclusion of a stated integer or groups of integers but not the exclusion of any other integer or group of integers.

The term “alkyl” refers to both straight and branched saturated chains containing, for example, 1-3, 1-6, 1-9, or 1-12 carbon atoms. An alkyl group may be optionally substituted. Substituents can include any substituents described herein, for example, but not limited to, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants.

The terms “artificial sweetener” and “sugar substitute” refer to a food additive that confers a sweet taste but has less caloric energy than sugar. In some instances, the caloric energy of the “artificial sweetener” or “sugar substitute” is negligible.

The term “bitter” or “bitter taste” as used herein refers to the perception or gustatory sensation resulting following the detection of a bitter tastant. The following attributes may contribute to bitter taste: astringent, bitter-astringent, metallic, bitter-metallic, as well as off-tastes, aftertastes and undesirable tastes including but not limited to freezer-burn and card-board taste, and/or any combinations of these. It is noted that, in the art, the term “off-taste” is often synonymous with “bitter taste.” Without being limited by theory, the diversity of bitter tastes may reflect the large number of bitter receptors and the differential detection of bitter tastants by these receptors. Bitter taste as used herein includes activation of a bitter taste receptor by a bitter tastant. Bitter taste as used herein also includes activation of a bitter taste receptor by a bitter tastant followed by downstream signaling. Bitter taste as used herein also includes activation of a signaling pathway after stimulation by a bitter tastant. Bitter taste as used herein further includes perception resulting from signaling following the detection of a bitter tastant by a bitter taste receptor. Bitter taste as used herein further includes perception resulting from signaling following contacting a bitter taste receptor with a bitter tastant. Bitter taste can be perceived in the brain.

The term “bitter taste receptor” refers to a receptor, typically a cell surface receptor, to which a bitter tastant can bind. Bitter taste receptors may be present in the oral cavity, and/or extra-oral tissues, e.g., in taste-like, hormone producing cells throughout the gastrointestinal tract, including the stomach, intestines, and colon. Bitter receptors can also be present in vitro, such as in an assay, including but not limited to a cell based assay or a binding assay.

The term “bitter tastant,” “bitter ligand,” or “bitter compound” refers to a compound that activates or that can be detected by a bitter taste receptor and/or confers the perception of a bitter taste in a subject. A “bitter tastant” also refers to a multiplicity of compounds that combine to activate or be detected by a bitter taste receptor and/or confer the perception of a bitter taste in a subject. A “bitter tastant” further refers to a compound that is enzymatically modified upon ingestion by a subject to activate or be detected by a bitter taste receptor and/or confer the perception of a bitter taste in a subject. Because the perception of bitter taste may vary from individual to individual, some individuals may describe a “bitter tastant” as a compound which confers a different kind of bitter taste compared to the kind of bitter taste perceived for the same compound by other individuals. The term bitter tastant also refers to a compound which confers a bitter taste. Those of skill in the art can readily identify and understand what is meant by a bitter tastant. Non-limiting examples of hitter tastants or substances including foods that comprise a bitter tastant and taste bitter include coffee, unsweetened cocoa, marmalade, bitter melon, beer, bitters, citrus peel, dandelion greens, escarole, quinine, magnesium salts, calcium salts, potassium salts, KCl, potassium lactate, acesulfame K, saccharin, rebaudioside A, rebaudioside C, stevioside, sucralose, tea polyphenols, Brussel sprouts, asparagus, hitter gourd, wild cucumber, celery, hops, kohlrabi, radish leaf; ginseng, pumpkin, collard greens, kale, sparteine, caffeine, atropine, nicotine, urea and strychnine.

Further examples of bitter tastants include pharmaceuticals. Non-limiting examples of pharmaceuticals as bitter tastants include acetaminophen, ampicillin, azithromycin, chlorpheniramine, cimetidine, dextromethorphan, diphenhydramine, erythromycin, esomeprazole, guaifenesin, ibuprofen, penicillin, phenylbutazone, pseudoephedrine, ranitidine, sildenafil, spironolactone and theophylline all of which have been associated with bitter taste.

The terms “combination” or “combinations” refer to mixture of two or more compounds of the invention. Combinations can include, but are not limited to, a combination of one or more compounds of Formula (1), or comestibly or biologically acceptable salts, derivatives, diastereomers, or enantiomers thereof; a combination of two or more comestibly or biologically acceptable salts, derivatives, or enantiomers of Compound 1; or a combination of Compound 1, or a comestibly or biologically acceptable salt, derivative, or enantiomer thereof, and one or more compounds of Formula (1), or comestibly or biologically acceptable salts, derivatives, diastereomers, or enantiomers thereof.

The term “comestibly or biologically acceptable salt” refers to any comestibly or biologically acceptable salt, ester, or salt of such ester, of a compound of the present invention, which, upon ingestion, is capable of providing (directly or indirectly) a compound of the present invention, or a metabolite, residue or portion thereof, characterized by the ability to reduce the perception of a bitter taste attributed to a bitter tastant. Similarly, the term “comestibly or biologically acceptable derivative” refers to any comestibly or biologically acceptable derivative of a compound of the present invention, which, upon ingestion, is capable of providing (directly or indirectly) a compound of the present invention, or a metabolite, residue or portion thereof, characterized by the ability to reduce the perception of a bitter taste attributed to a bitter tastant. A “comestible product” is a product suitable for oral use, such as eating or drinking. Therefore, a comestibly acceptable compound is an edible compound.

The term “consumer product” refers to health and beauty products for the personal use and/or consumption by a subject. Consumer products may be present in any form including, but not limited to, liquids, solids, semi-solids, tablets, capsules, lozenges, strips, powders, gels, gums, pastes, slurries, syrups, aerosols and sprays. Non-limiting examples of consumer products include nutriceuticals, nutritional supplements, lipsticks, lip balms, soaps, shampoos, gums, adhesives (e.g., dental adhesives), toothpastes, oral analgesics, breath fresheners, mouthwashes, tooth whiteners, and other dentifrices.

The term “diet” collectively refers to the food products and/or beverages consumed by a subject. A subject's “diet” also includes any consumer products or pharmaceutical compositions the subject ingests.

The term “edible composition” refers to a composition suitable for consumption, typically via the oral cavity (although consumption may occur via non-oral means such as inhalation). Edible compositions may be present in any form including, but not limited to, liquids, solids, semi-solids, tablets, lozenges, powders, gels, gums, pastes, slurries, syrups, aerosols and sprays. As used herein, edible compositions include food products, pharmaceutical compositions, and consumer products. The term edible compositions also refers to, for example, dietary and nutritional supplements. As used herein, edible compositions also include compositions that are placed within the oral cavity but not swallowed, including professional dental products, such as dental treatments, fillings, packing materials, molds and polishes. The term “comestible” refers to similar compositions and is generally used as a synonym to the term “edible.”

The term “effective amount” refers to an amount sufficient to produce a desired property or result. For example, an effective amount of a compound of the present invention is an amount capable of reducing the perception of bitter taste associated with a bitter tastant. The term “effective amount” of a compound of the invention also refers to an amount which, when added to an edible composition, reduces the bitter taste of, e.g., a sugar substitute, thereby allowing for the maintenance of the perception of a desired sweet flavor of a said edible composition. The term “effective amount of a compound” also refers to an amount which, when added to an edible composition, allows for the preservation of a food product, while reducing or eliminating bitter taste associated with a bitter tastant in the preservative. The term “effective amount” also refers to the amount of a compound of the present invention capable or reducing or eliminating the perception of a hitter taste or aftertaste associated with either a bitter tastant in a food product or an inherently bitter food product.

The term “flavor modifier” refers to a compound or a mixture of compounds that, when added to an edible composition, such as a food product, modifies (e.g., masks, eliminates, decreases, reduces, or enhances the perception of) a flavor (e.g., sweet, salty, umami, sour, or hitter taste) present in the edible composition.

The term “food product” refers to any composition comprising one or more processed foodstuff. Food products include, but are not limited to, confectionaries, bakery products (including, but not limited to, doughs, breads, biscuits, crackers, cakes, pastries, pies, tarts, quiches, and cookies), ice creams (including but not limited to impulse ice cream, take-home ice cream, frozen yogurt, gelato, sorbet, sherbet and soy, oat, bean and rice-based ice cream), dairy products (including, but not limited to, drinking milk, cheese, yogurt, and sour milk drinks), cheeses (including, but not limited to, natural cheeses and processed cheeses), butter, margarine, sweet and savory snacks (including but not limited to fruit snacks, chips/crisps, tortilla/corn chips, popcorn, pretzels, chocolates, and nuts), hot and cold beverages (including, but not limited to, beverages, beverage mixes, concentrates, juices, carbonated beverages, non-carbonated beverages, alcoholic beverages, non-alcoholic beverages, soft drinks, sports drinks, isotonic drinks, coffees, teas, bottled waters, and beverages prepared from botanicals and botanical extracts (including cold beverages that are prepared with botanical or fungi extracts as ingredients, and drinks that are prepared in various ways, such as intisions, decoctions, or other means of extraction or distillation of various plant parts, including, but not limited to leaves, flowers, stems, fruits, roots, rhizomes, stems, bark, volatile oils, or even the whole plant)), snack bars (including, but not limited to granola bars, muesli bars, protein bars, breakfast bars, energy bars, and fruit bars), meal replacement products, ready meals (including, but not limited to canned meals, preserved meals, frozen meals, dried meals, chilled meals, dinner mixes and prepared salads), soups (including but not limited to broth-like soups and cream-based soups), broth, gravy, soy sauce, meats and fish (including raw, cooked, and dried meats), deli products (including but not limited to meats and cheeses suitable for slicing or pre-sliced meats and cheeses, e.g., turkey, chicken, ham, bologna, salami, bierwurst, capicola, chorizo, corned beef, Dutch loaf, Serrano ham, prosciutto, head cheese, liverwurst, meatloaf (including olive loaf, pepper loaf, pimento loaf, and ham and cheese loaf), mortadella, pastrami, pepperoni, roast beef, roast pork, saucisson, smoked meat, summer sausage, tongue, American cheese, blue cheese, cheddar cheese, Colby cheese, Colby-Jack cheese, gouda, Monterey Jack cheese, Muenster cheese, mozzarella, Parmigiano cheese, pepper jack cheese, provolone, Romano cheese, string cheese, spray cheese, and Swiss cheese), vegetables (including, but not limited to, raw, pickled, cooked, and dried vegetables, such as french fries), fruits (including raw, processed, cooked, and dried fruits), grains (including, but not limited to, dried cereals and breads), prepared foods (including, but not limited to, dried, canned, or jarred sauces and soups), snack foods, pastas (including, but not limited to, fresh pasta, chilled pasta, frozen pasta, dried pasta, and macaroni), noodles (including, but not limited to, egg noodles, wheat noodles, rice noodles, mung bean noodles, potato noodles, buckwheat noodles, corn noodles, cellophane noodles, chow mein, fettuccini, fusilli, gnocchi, lasagna, linguini, lo mein, macaroni, manicotti, pad thai, penne, ramen, rice vermicelli, rigatoni, soba, spaghetti, spatzle, udon, and ziti), canned foods, frozen foods, dried foods, chilled foods, oils and fats, baby food, spreads, salads, cereals (including, but not limited to, hot and cold cereals), sauces (including, but not limited to, cheese sauces, tomato pastes, tomato purees, bouillon cubes, stock cubes, table sauces, bouillabaisse sauces, pasta sauces, cooking sauces, marinades, dry sauces, powder mixes, ketchups, mayonnaises, salad dressings, vinaigrettes, mustards, and dips), jellies, jams, preserves, honey, puddings, recipe mixes, syrups, icings, fillings, infused foods, soft candies, sugar substitutes, salt-preserved food, marinated foods and condiments (such as ketchup, mustard and steak sauce). In some embodiments, the food product is animal feed. For example, the food product may be a pet food product, i.e., a food product for consumption by a household pet. In some embodiments, the food product is a livestock food product, i.e., a food product for consumption by livestock.

The term “foodstuff” refers to an unprocessed ingredient or a basic nutrient or flavor containing element used to prepare a food product. Non-limiting examples of foodstuffs include: fruits, vegetables, meats, fishes, grains, milks, eggs, tubers, sugars, sweeteners, oils, herbs, snacks, sauces, spices and salts.

The term “high potency sweetener” means a synthetic or artificial high potency sweetener and a natural high-potency sweetener.

The terms “natural high-potency sweetener,” “NHPS,” “NHPS composition.” and “natural high-potency sweetener composition” are used interchangeably, herein, and refer to any sweetener found in nature which may be in raw, extracted, purified, or any other form, singularly or in combination thereof and characteristically have a sweetness potency greater than sucrose, fructose, or glucose, yet have fewer or no calories. Non-limiting examples of NHPSs suitable for embodiments of this disclosure include steviol glycoside, rebaudioside A, rebaudioside B, rebaudioside C (dulcoside B), rebaudioside D, rebaudioside E, rebaudioside F, rebaudioside I, rebaudioside H, rebaudioside L, rebaudioside K, rebaudioside J, rebaudioside N, rebaudioside O, rebaudioside M, dulcoside A, rubusoside, stevia leaf extract, stevioside, glycosylated steviol glycosides, mogrosides, mogroside V, isomogroside, mogroside IV, Luo Han Guo fruit extract, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pitrocaryoside B, mukurozioside, phlomisoside I, periandrin I, abrusoside A, or cyclocarioside I. NHPS also includes modified NHPSs. Modified NHPSs include NHPSs which have been altered naturally. For example, a modified NHPS includes, but is not limited to, NHPSs which have been fermented, contacted with enzyme, or derivatized or substituted on the NHPS. In one embodiment, at least one modified NHPS may be used in combination with at least one NHPS. In another embodiment, at least one modified NHPS may be used without a NHPS. Thus, modified NHPSs may be substituted for a NHPS or may be used in combination with NHPSs for any of the embodiments described herein. For the sake of brevity, however, in the description of embodiments, a modified NHPS is not expressly described as an alternative to an unmodified NHPS, but it should be understood that modified NHPSs can be substituted for NHPSs in any embodiment disclosed herein.

The terms “parts per million,” “ppm,” “parts per billion,” and “ppb” are used in the food industry to refer to a low concentration of a solution. For example, one gram of solute in 1000 mL of solvent has a concentration of 1000 ppm and one thousandth of a gram (0.001 g) of solute in 1000 mL of solvent has a concentration of one ppm. Accordingly, a concentration of one milligram per liter (i.e., 1 mg/L) is equal to 1 ppm, 0.001 gram of solute in 1000 mL of solvent has a concentration of 1000 ppb and a concentration of 0.001 milligram per liter (i.e., 0.001 mg/L) is equal to 1 ppb.

The terms “perception of a bitter taste.” “perception of sweetness,” “perception of a flavor” and similar terms, refer to the awareness of a subject of a particular taste or flavor.

The term “pharmaceutically active ingredient” refers to a compound in a pharmaceutical composition which is biologically active.

The term “processed foodstuff” refers to a foodstuff has been subjected to any process which alters its original state (excluding, e.g., harvesting, slaughtering, and cleaning). Examples of methods of processing foods include, but are not limited to, removal of unwanted outer layers, such as potato peeling or the skinning of peaches; chopping or slicing; mincing or macerating; liquefaction, such as to produce fruit juice; fermentation (e.g., beer); emulsification; cooking, such as boiling, broiling, frying, heating, steaming or grilling; deep frying; baking; mixing; addition of gas such as air entrainment for bread or gasification of soft drinks; proofing; seasoning (with, e.g., herbs, spices, salts); spray drying; pasteurization; packaging (e.g., canning or boxing); extrusion; puffing; blending; and preservation (e.g., adding salt, sugar, potassium lactate or other preservatives).

The term “replace” or “replacing” refers to substituting one compound for another compound in or in the preparation of, for example, an edible composition, such as food product. It includes complete and partial replacements or substitutions.

The term “stability” or “stable” in the context of a chemical structure refers to the chemical state when a system is in its lowest energy state, or in chemical equilibrium with its environment. Thus, a stable compound (or, e.g., a compound containing a number of atoms or substitutions that are stable) is not particularly reactive in the environment or during normal use, and retains its useful properties on the timescale of its expected usefulness.

The term “subject” refers to a mammal. In preferred embodiments, the subject is human. In some embodiments, a subject is a domestic or laboratory animal, including but not limited to, household pets, such as dogs, cats, pigs, rabbits, rats, mice, gerbils, hamsters, guinea pigs, and ferrets. In some embodiments, a subject is a livestock animal. Non-limiting examples of livestock animals include: alpaca, bison, camel, cattle, deer, pigs, horses, llamas, mules, donkeys, sheep, goats, rabbits, reindeer, and yak.

The term “sugar” refers to a simple carbohydrate, such as a monosaccharide or a disaccharide, that delivers a primary taste sensation of sweetness. Non-limiting examples of sugar include glucose, fructose, galactose, sucrose, lactose, and maltose.

The term “sweet flavor” refers to the taste elicited by, for example, sugars. Non-limiting examples of compositions eliciting a sweet flavor include glucose, sucrose, fructose, saccharin, cyclamate, aspartame, acesulfame potassium, sucralose, alitame, and neotame. The amount of sweet flavor or the sweetness of a composition can be determined by, e.g., taste testing.

The terms “synthetic high potency sweetener” and “artificial high potency sweetener” are used interchangeably herein and refer to any composition which is not found naturally in nature and characteristically has a sweetness potency greater than sucrose, fructose, or glucose, yet have fewer or no calories. Non-limiting examples of synthetic sweeteners suitable for embodiments of this invention include sucralose, acesulfame potassium or other salts, aspartame, alitame, saccharin, neohesperidin dihydrochalcone, cyclamate, neotame, advantame, and salts thereof.

As defined herein, the compounds of the invention are intended to include all stereochemical forms of the compound, including geometric isomers (i.e., E, Z) and optical isomers (i.e., R, S). Single stereochemical isomers as well as enantiomeric and diastereomeric mixtures of the present compounds are within the scope of the invention. Unless otherwise stated, formulas depicted herein are also meant to include compounds which differ only in the presence of one or more isotopically enriched atoms. For example, compounds having the present formulas except for the replacement of a hydrogen by a deuterium or tritium, or the replacement of a carbon by a ¹³C- or ¹⁴C-enriched carbon are within the scope of this invention.

The present invention provides edible compositions comprising a compound of the present invention, including food products, consumer products, and pharmaceutical compositions comprising said compounds, and methods of preparing a such compositions. The present invention also provides methods of reducing the amount of sugar in a food product, a method of reducing bitter taste, and a method of reducing the lingering sweetness or aftertaste of a high potency sweetener. High potency sweeteners also can be bitter tastants. Non limiting examples include aspartame, acesulfame K, saccharin, stevioside, rebaudioside A, and rebaudioside C. While providing sweetness in calorie reduced foods, high potency sweeteners have a bitter aftertaste and/or a time intensity profile that differs from sugars. This unfavorable sensory profile reduces the utility of high potency sweeteners. It would be advantageous to use the compounds or preparations of the invention to mask the bitterness of and improve the taste of high potency sweeteners.

### Sweet Taste Improving Compositions

The terms “sweet taste improving composition” and “sweet taste improving additive” are used interchangeably herein and refer to any material that imparts a more sugar-like temporal profile or sugar-like flavor profile or both to a synthetic sweetener (i.e., corrects linger). Suitable sweet taste improving additives useful in embodiments of this disclosure include amino acids and salts thereof, poly-amino acids and salts thereof, peptides, sugar acids and salts thereof, nucleotides and salts thereof, organic acids, inorganic acids, organic salts including organic acid salts and organic base salts, inorganic acid salts (e.g., sodium chloride, potassium chloride, magnesium chloride), acid salts (e.g., sodium citrate), bitter compounds, flavorants and flavoring ingredients, astringent compounds, polymers, proteins or protein hydrolysates, surfactants, emulsifiers, flavonoids, alcohols, and natural high-potency sweeteners.

The terms “sugar-like characteristic,” “sugar-like taste,” “sugar-like sweet,” “sugary,” and “sugar-like” are used interchangeably, herein, and include any characteristic similar to that of sucrose and include, but are not limited to, maximal response, flavor profile, temporal profile, adaptation behavior, mouth feel, concentration/response function behavior, tastant and flavor/sweet taste interactions, spatial pattern selectivity, and temperature effects. These characteristics are dimensions in which the taste of sucrose is different from the tastes of sweetness enhanced sweetener compositions. Suitable procedures for determining whether a composition has a more sugar-like taste are well known in the art.

The compositions of the present invention may also further comprise at least one additional additive, such as a sweet taste improving composition or a sweet taste improving additive. For example, the composition of the disclosure may comprise at least one sweet taste improving composition for balancing the temporal and/or flavor profile of sweet compositions. The use of sweet taste improving compositions to improve the temporal and/or flavor profile of sweetener compositions are described in detail in U.S. Patent Application Publication Nos. 2007/0128311, 2007/0275147, 2008/0292765, 2011/0160311, and 2011/0318464 the disclosures of which are incorporated herein by reference in their entirety.

Exemplary suitable sweet-taste improving compounds include, but are not limited to, carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, flavonoids, alcohols, polymers, other sweet taste improving taste additives imparting such sugar-like characteristics, and combinations thereof. In some embodiments, the sweet-taste improving compound is erythritol. Erythritol is sometimes used in a range of 0.5 ppm to 3.5 ppm

Suitable sweet taste improving amino acid additives for use in embodiments of this disclosure include, but are not limited to, aspartic acid, arginine, glycine, glutamic acid, proline, threonine, theanine, cysteine, cystine, alanine, valine, tyrosine, leucine, isoleucine, asparagine, serine, lysine, histidine, ornithine, methionine, carnitine, aminobutyric acid (α-, β-, or γ-isomers), glutamine, hydroxyproline, taurine, norvaline, sarcosine, and their salt forms such as sodium or potassium salts or acid salts. The sweet taste improving amino acid additives also may be in the D- or L-configuration and in the mono-, di-, or tri-form of the same or different amino acids. Additionally, the amino acids may be α-, β-, γ-, δ-, and ε-isomers if appropriate. Combinations of the foregoing amino acids and their corresponding salts (e.g., sodium, potassium, calcium, magnesium salts or other alkali or alkaline earth metal salts thereof, or acid salts) also are suitable sweet taste improving additives in some embodiments. The amino acids may be natural or synthetic. The amino acids also may be modified. Modified amino acids refers to any amino acid wherein at least one atom has been added, removed, substituted, or combinations thereof (e.g., N-alkyl amino acid, N-acyl amino acid, or N-methyl amino acid). Non-limiting examples of modified amino acids include amino acid derivatives such as trimethyl glycine, N-methyl-glycine, and N-methyl-alanine. As used herein, modified amino acids encompass both modified and unmodified amino acids. As used herein, amino acids also encompass both peptides and polypeptides (e.g., dipeptides, tripeptides, tetrapeptides, and pentapeptides) such as glutathione and L-alanyl-L-glutamine. Suitable sweet taste improving polyamino acid additives include poly-L-aspartic acid, poly-L-lysine (e.g., poly-L-α-lysine or poly-L-ε-lysine), poly-L-ornithine (e.g., poly-L-α-ornithine or poly-L-ε-ornithine), poly-L-arginine, other polymeric forms of amino acids, and salt forms thereof (e.g., calcium, potassium, sodium, or magnesium salts such as L-glutamic acid mono sodium salt). The sweet taste improving poly-amino acid additives also may be in the D- or L-configuration. Additionally, the poly-amino acids may be α-, β-, γ-, δ-, and ε-isomers if appropriate. Combinations of the foregoing poly-amino acids and their corresponding salts (e.g., sodium, potassium, calcium, magnesium salts or other alkali or alkaline earth metal salts thereof or acid salts) also are suitable sweet taste improving additives in some embodiments. The poly-amino acids described herein also may comprise co-polymers of different amino acids. The poly-amino acids may be natural or synthetic. The poly-amino acids also may be modified, such that at least one atom has been added, removed, substituted, or combinations thereof (e.g., N-alkyl poly-amino acid or N-acyl poly-amino acid). As used herein, poly-amino acids encompass both modified and unmodified poly-amino acids. For example, modified poly-amino acids include, but are not limited to poly-amino acids of various molecular weights (MW), such as poly-L-α-lysine with a molecular weight of about 1,500, 6,000, 25,200, 63,000, 83,000, or 300,000. In some embodiments, the poly-amino acids have a molecular weight of 1,500, 6,000, 25,200, 63,000, 83,000, or 300,000. In some embodiments, the taste improving amino acid additive is glycine, alanine, taurine, serine or proline. In such embodiments, the taste improving amino acid additive is present in a concentration of about 10 ppm to about 25,000 ppm or about 100 to about 1000 ppm. In other such embodiments, the taste improving amino acid additive is present in a concentration of 10 ppm to 25,000 ppm or 100 to 1000 ppm.

Suitable sweet taste improving sugar acid additives include, for example, but are not limited to aldonic, uronic, aldaric, alginic, gluconic, glucuronic, glucaric, galactaric, galacturonic, and salts thereof (e.g., sodium, potassium, calcium, magnesium salts or other physiologically acceptable salts), and combinations thereof.

For example, suitable sweet taste improving nucleotide additives include, but are not limited to, inosine monophosphate (“IMP”), guanosine monophosphate (“GMP”), adenosine monophosphate (“AMP”), cytosine monophosphate (“CMP”), uracil monophosphate (“UMP”), inosine diphosphate, guanosine diphosphate, adenosine diphosphate, cytosine diphosphate, uracil diphosphate, inosine triphosphate, guanosine triphosphate, adenosine triphosphate, cytosine triphosphate, uracil triphosphate, alkali or alkaline earth metal salts thereof, and combinations thereof. The nucleotides described herein also may comprise nucleotide-related additives, such as nucleosides or nucleic acid bases (e.g., guanine, cytosine, adenine, thymine, or uracil).

Suitable sweet taste improving organic acid additives include any compound which comprises a —COOH moiety. Suitable sweet taste improving organic acid additives, for example, include but are not limited to C₂-C₃₀ carboxylic acids, substituted hydroxyl C₂-C₃₀ carboxylic acids, benzoic acid, substituted benzoic acids (e.g., 2,4-dihydroxybenzoic acid), substituted cinnamic acids, hydroxyacids, substituted hydroxybenzoic acids, substituted cyclohexyl carboxylic acids, tannic acid, lactic acid, tartaric acid, citric acid, gluconic acid, glucoheptonic acids, adipic acid, hydroxycitric acid, malic acid, fruitaric acid (a blend of malic, fumaric, and tartaric acids), fumaric acid, maleic acid, succinic acid, chlorogenic acid, salicylic acid, creatine, caffeic acid, bile acids, acetic acid, ascorbic acid, alginic acid, erythorbic acid, polyglutamic acid, glucono delta lactone, and their alkali or alkaline earth metal salt derivatives thereof. In addition, the organic acid additives also may be in either the D- or L-configuration.

For example, suitable sweet taste improving organic acid additive salts include, but are not limited to, sodium, calcium, potassium, and magnesium salts of all organic acids, such as salts of citric acid, malic acid, tartaric acid, fumaric acid, lactic acid (e.g., sodium lactate), alginic acid (e.g., sodium alginate), ascorbic acid (e.g., sodium ascorbate), benzoic acid (e.g., sodium benzoate or potassium benzoate), and adipic acid. The examples of the sweet taste improving organic acid additives described optionally may be substituted with at least one group chosen from hydrogen, alkyl, alkenyl, alkynyl, halo, haloalkyl, carboxyl, acyl, acyloxy, amino, amido, carboxyl derivatives, alkylamino, dialkylamino, arylamino, alkoxy, aryloxy, nitro, cyano, sulfo, thiol, imine, sulfonyl, sulfenyl, sulfinyl, sulfamyl, carboxalkoxy, carboxamido, phosphonyl, phosphinyl, phosphoryl, phosphino, thioester, thioether, anhydride, oximino, hydrazino, carbamyl, phospho, phosphonato, and any other viable functional group provided the substituted organic acid additives function to improve the sweet taste of a synthetic sweetener.

For example, suitable sweet taste improving inorganic acid additives include but are not limited to phosphoric acid, phosphorous acid, polyphosphoric acid, hydrochloric acid, sulfuric acid, carbonic acid, sodium dihydrogen phosphate, and alkali or alkaline earth metal salts thereof (e.g., inositol hexaphosphate Mg/Ca).

Suitable sweet taste improving bitter compound additives, for example, include but are not limited to caffeine, quinine, urea, bitter orange oil, naringin, quassia, and salts thereof.

### Edible Compositions

According to one aspect, the invention provides an edible composition comprising a compound of the invention for reducing bitter taste of a bitter tastant.

All stereochemical forms of the compounds disclosed in this and any section herein are specifically contemplated, including geometric isomers (i.e., E, Z) and optical isomers (i.e., R, S). Single stereochemical isomers as well as enantiomeric and diastereomeric mixtures of the compounds disclosed in this and any section herein are also specifically contemplated.

In some embodiments, the present invention provides an edible composition for reducing bitter taste of a bitter tastant, wherein the composition comprises a modified amino acid compound. The modified amino acid compounds of this invention are capable of reducing or eliminating bitter taste of a bitter tastant. In some embodiments, the modified amino acid compound has a molecular weight less than about 2000, 1500, 1000, 500, or 300 daltons. In some embodiments, the modified amino acid compound has a molecular weight less than 2000, 1500, 1000, 500, or 300 daltons.

In some embodiments, the present invention provides an edible composition for reducing bitter taste of a bitter tastant, wherein the composition comprises a compound of Formula (I), or a comestibly or biologically acceptable salt, derivative, enantiomer, or diastereomer thereof, or combinations of any of the aforementioned. The compound of Formula (I), or a comestibly or biologically acceptable salt, derivative, enantiomer, or diastereomer thereof, or combinations of any of the aforementioned, is capable of reducing or eliminating bitter taste of a bitter tastant. In some embodiments, the compound of Formula (I), or a comestibly or biologically acceptable salt, derivative, enantiomer, or diastereomer thereof, has a molecular weight less than about 2000, 1500, 1000, 500, or 300 daltons. In some embodiments, the compound of Formula (I), or a comestibly or biologically acceptable salt, derivative, enantiomer, or diastereomer thereof, has a molecular weight less than 2000, 1500, 1000, 500, or 300 daltons. In certain embodiments, the compound of Formula (I) is:

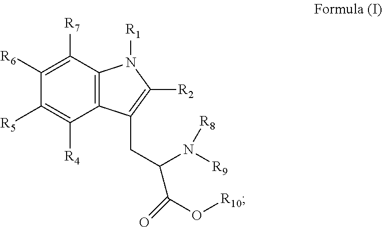

or a comestibly or biologically acceptable salt, derivative, diastereomer, or enantiomer thereof,
wherein, as valance and stability permit:
R₁ is independently H or C₁-C₆ alkyl;
R₂ is independently H or C₁-C₆ alkyl;
R₄ is independently H, OH, C₁-C₆ alkyl, or O(C₁-C₆) alkyl;
R₅ is independently H, OH, C₁-C₆ alkyl, or O(C₁-C₆) alkyl;
R₆ is independently H, OH, C₁-C₆ alkyl, or O(C₁-C₆) alkyl;
R₇ is independently H, OH, C₁-C₆ alkyl, or O(C₁-C₆) alkyl;
R₈ is independently H. C₁-C₆ alkyl, C(O)C₁-C₆ alkyl, C(O)R₁₁, or R₁₂;
R₉ is independently H, C₁-C₆ alkyl, C(O)C₁-C₆ alkyl, C(O)R₁₁, or R₁₂;
R₁₀ is independently H or C₁-C₆ alkyl;

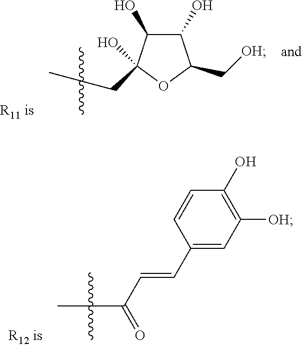 [SMILES: *C[C@]1(O)O[C@H](CO)[C@@H](O)[C@@H]1O ; *C(=O)/C=C/c1ccc(O)c(O)c1]

wherein the composition is edible and capable of reducing bitter taste of a bitter tastant.

According to some embodiments of compounds of Formula (I), or a comestibly or biologically acceptable salt, derivative, or enantiomer thereof, wherein, as valence and stability permit:
R₁ is independently H or C₁-C₆ alkyl;
R₂ is independently H or C₁-C₆ alkyl;
R₄ is independently H, OH, C₁-C₆ alkyl, or O(C₁-C₆) alkyl;
R₅ is independently H, OH, C₁-C₆ alkyl, or O(C₁-C₆) alkyl;
R₆ is independently H, OH, C₁-C₆ alkyl, or O(C₁-C₆) alkyl;
R₇ is independently H, OH, C₁-C₆ alkyl, or O(C₁-C₆) alkyl;
R₈ is independently H, C₁-C₆ alkyl, or C(O)C₁-C₆ alkyl;
R₉ is independently H, C₁-C₆ alkyl, or C(O)C₁-C₆ alkyl; and
R₁₀ is independently H or C₁-C₆ alkyl.

According to some embodiments of compounds of Formula (I), or a comestibly or biologically acceptable salt, derivative, or enantiomer thereof, wherein, as valence and stability permit:
R₁ is H;
R₂ is H;
R₄ is independently H, OH, or O(C₁-C₆) alkyl;
R₅ is independently H, OH, or O(C₁-C₆) alkyl;
R₆ is independently H, OH, or O(C₁-C₆) alkyl;
R₇ is independently H, OH, or O(C₁-C₆) alkyl;
R₈ is independently H or C₁-C₆ alkyl;
R₉ is independently H or C₁-C₆ alkyl; and
R₁₀ is independently H or C₁-C₆ alkyl.

According to some embodiments of compounds of Formula (I), or a comestibly or biologically acceptable salt, derivative, or enantiomer thereof, wherein, as valence and stability permit:
R₁ is H; R₂ is H; R₄ is H; R₅ is H; R₆ is H; R₇ is H; R₈ is H; R₉ is C₁-C₆ alkyl; and R₁₀ is H.

In certain embodiments, the compound of Formula (I) is:

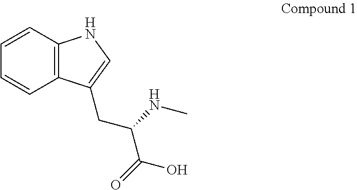 [SMILES: CN[C@@H](Cc1c[nH]c2ccccc12)C(=O)O]

or a comestibly or biologically acceptable salt, derivative, or enantiomer thereof.

If a comestibly or biologically acceptable salt of a compound of the present invention is used, such salt is preferably derived from inorganic or organic acids and bases. Examples of such salts include, but are not limited to, those derived from appropriate bases, including alkali metal (e.g., sodium and potassium), alkaline earth metal (e.g., magnesium), ammonium and N⁺(C₁₋₄ alkyl)₄ salts.

Another aspect of the present invention provides edible compositions comprising a) a compound of the invention; and b) a bitter tastant. In some embodiments, the compound is a compound having a molecular weight less than about 1500, 1000, 500, or 300 daltons. In some embodiments, the compound is a compound having a molecular weight less than 1500, 1000, 500, or 300 daltons. In certain embodiments, the compound is a compound of Formula (I), or a comestibly or biologically acceptable salt, derivative, enantiomer, or diastereomer thereof, as described herein. In some embodiments, the compound of the invention is Compound 1, or a comestibly or biologically acceptable salt, derivative, or enantiomer thereof, as described herein.

In some embodiments, the edible composition further comprises one or more additional components selected from the group consisting of preservatives, nutritives, flavorants or additional flavor modifiers, including sweet taste modifying compositions which may lack an inherent flavor.

In some embodiments, the edible composition further comprises one or more emulsifiers. Sodium and potassium based emulsifiers are commonly used as emulsifiers in the food art. Sodium-based emulsifiers include, e.g., sodium salts of fatty acids, sodium alginate, sodium aluminum phosphate, sodium caseinate, sodium metaphosphate, sodium phosphate (dibasic), sodium phosphate (monobasic), sodium phosphate (tribasic), sodium polyphosphate, sodium pyrophosphate, and sodium stearoyl lactylate. Potassium-based emulsifiers include, e.g., potassium salts of fatty acids, potassium alginate, potassium citrate, potassium phosphate (dibasic), potassium phosphate (monobasic), potassium phosphate (tribasic), potassium polyphosphate, potassium polymetaphosphate, and potassium pyrophosphate. Accordingly, some embodiments of the present invention include replacing a sodium-based emulsifier with a potassium based emulsifier and adding a compound of the present invention.

In some embodiments, the edible composition further comprises a surfactant to increase or decrease the effectiveness of the compounds of the present invention. Suitable surfactants include, but are not limited to, non-ionic surfactants (e.g., mono and diglycerides, fatty acid esters, sorbitan esters, propylene glycol esters, and lactylate esters) anionic surfactants (e.g., sulfosuccinates and lecithin) and cationic surfactants (e.g., quaternary ammonium salts).

In some embodiments wherein the edible compositions further comprises a preservative, the preservative improves the shelf life of the edible composition. Suitable preservatives include, but are not limited to, ascorbic acid, benzoic acid, butyl p-hydroxybenzoate, calcium benzoate, calcium disodium EDTA, calcium hydrogen sulfite, calcium propionate, calcium sorbate, chitosan, cupric sulfate, dehydroacetic acid, diethyl pyrocarbonate, dimethyl dicarbonate, disodium EDTA, E-polylysine glycine, erythorbic acid, ethyl p-hydroxybenzoate, formic acid, gum guaiac, heptylparaben, hinokitiol, isobutyl paraoxybenzoate, Japanese styrax benzoin extract, methylparaben, milt protein extract, natamycin, nisin, peptin extract, 2-phenylphenol, pimaricin, potassium acetate, potassium benzoate, potassium lactate, potassium metabisulfite, potassium nitrate, potassium nitrite, potassium pyrosulfite, potassium sorbate, potassium sulfite, propionic acid, propyl p-hydroxybenzoate, propyl p-oxybenzoate, propylene oxide, propylparaben, sodium benzoate, sodium bisulfite, sodium dehydroacetate, sodium diacetate, sodium erythorbate, sodium hydrogen sulfite, sodium hypophosphite, sodium hyposulfite, sodium metabisulfite, sodium nitrate, sodium nitrite, sodium o-phenylphenol, sodium propionate, sodium pyrosulfite, sodium sulfite, sodium thiocyanate, sorbic acid and sulfur dioxide. In some embodiments, the preservative has a bitter flavor.

In some embodiments, the composition may further comprise one or more additional components selected from the group consisting of flow agents, processing agents, sugars, amino acids, other nucleotides, and sodium or potassium salts of organic acids such as citrate and tartarate. Such additional ingredients may add flavor, or aid in blending, processing or flow properties of the edible composition.

In some embodiments, the rate of release of the compound of the present invention is regulated. The release rate of the compound of the present invention can be altered by, for example, varying its solubility in water. Rapid release can be achieved by encapsulating the compound of the present invention with a material with high water solubility. Delayed release of the compound of the present invention can be achieved by encapsulating the compound of the present invention with a material with low water solubility. The compound of the present invention can be co-encapsulated with carbohydrates or masking tastants such as sweeteners. The rate of release of the compound of the present invention can also be regulated by the degree of encapsulation. In some embodiments, the compound of the present invention is fully encapsulated. In some embodiments, the compounds of the present invention are partially encapsulated. In some embodiments, the rate of release is regulated so as to release with the bitter tastant.

The edible compositions of this invention are prepared according to techniques well-known in the art. In general an edible composition of the invention is prepared by mixing a component or ingredient of the edible composition with a compound of the invention. Alternatively, a compound of the invention can be added directly to the edible composition. In some embodiments, a bitter tastant is added simultaneously or sequentially with a compound of the invention. If sequentially, the hitter tastant may be added before or after the compound of the invention. In some embodiments, the edible composition is a food product. In some embodiments, the edible composition is a pharmaceutical composition. In some embodiments, the edible composition is a consumer product.

The amount of both a compound of the present invention and a hitter tastant used in an edible composition depends upon a variety of factors, including the purpose of the composition and the desired or acceptable perception of bitterness or sweetness. The amount may depend on the nature of the edible composition, the particular compound added, the bitter tastant, other compounds present in the composition, the method of preparation (including amount of heat used), and the pH of the edible composition. It will be understood that those of skill in the art will know how to determine the amounts needed to produce the desired taste(s).

In general, a compound of the present invention in an edible composition may be present at a concentration between about 0.001 ppm and 1000 ppm. In some embodiments, the edible composition comprises between about 0.005 to 500 ppm; 0.01 to 100 ppm; 0.05 to 50 ppm; 0.1 to 5 ppm; 0.1 to 10 ppm; 1 to 10 ppm; 1 to 30 ppm; 1 to 50 ppm; 10 to 30 ppm; 10 to 50 ppm; or 30 to 50 ppm of a compound of the present invention. In yet further embodiments, the edible composition comprises about 0.1 to 30 ppm, 1 to 30 ppm or 1 to 50 ppm of a compound of the present invention. In additional embodiments, the edible composition comprises about 0.001 to 5 ppm, 0.005 to 5 ppm, 0.01 to 5 ppm, 0.05 to 5 ppm, 0.1 to 5 ppm; 0.1 to 4 ppm; 0.1 to 3 ppm; 0.1 to 2 ppm; 0.1 to 1 ppm; 0.5 to 5 ppm; 0.5 to 4 ppm; 0.5 to 3 ppm; 0.5 to 2 ppm; 0.5 to 1.5 ppm; 0.5 to 1 ppm; 5 to 15 ppm; 6 to 14 ppm; 7 to 13 ppm; 8 to 12 ppm; 9 to 11 ppm; 25 to 35 ppm; 26 to 34 ppm; 27 to 33 ppm; 28 to 32 ppm; or 29 to 31 ppm.

In some embodiments, a compound of the present invention in an edible composition may be present at a concentration between 0.001 ppm and 1000 ppm. In some embodiments, the edible composition comprises between 0.005 to 500 ppm; 0.01 to 100 ppm; 0.05 to 50 ppm; 0.1 to 5 ppm; 0.1 to 10 ppm; 1 to 10 ppm; 1 to 30 ppm; 1 to 50 ppm; 10 to 30 ppm; 10 to 50 ppm; or 30 to 50 ppm of a compound of the present invention. In yet further embodiments, the edible composition comprises 0.1 to 30 ppm, 1 to 30 ppm or 1 to 50 ppm of a compound of the present invention. In additional embodiments, the edible composition comprises 0.001 to 5 ppm, 0.005 to 5 ppm, 0.01 to 5 ppm, 0.05 to 5 ppm, 0.1 to 5 ppm; 0.1 to 4 ppm; 0.1 to 3 ppm; 0.1 to 2 ppm; 0.1 to 1 ppm; 0.5 to 5 ppm; 0.5 to 4 ppm; 0.5 to 3 ppm; 0.5 to 2 ppm; 0.5 to 1.5 ppm; 0.5 to 1 ppm; 5 to 15 ppm; 6 to 14 ppm; 7 to 13 ppm; 8 to 12 ppm; 9 to 11 ppm; 25 to 35 ppm; 26 to 34 ppm; 27 to 33 ppm; 28 to 32 ppm; or 29 to 31 ppm.

In yet further embodiments, the edible composition comprises about 0.001 ppm, about 0.005 ppm, about 0.01 ppm, about 0.05 ppm, about 0.1 ppm, about 0.5 ppm, about 1 ppm, about 2 ppm, about 3 ppm, about 4 ppm, about 5 ppm, about 6 ppm, about 7 ppm, about 7.5 ppm, about 8 ppm, about 9 ppm, or about 10 ppm of a compound of the present invention. In some embodiments, the edible composition comprises about 11 ppm, about 12 ppm, about 13 ppm, about 14 ppm, about 15 ppm, about 16 ppm, about 17 ppm, about 18 ppm, about 19 ppm, about 20 ppm, about 21 ppm, about 22 ppm, about 23 ppm, about 24 ppm, about 25 ppm, about 26 ppm, about 27 ppm, about 28 ppm, about 29 ppm, or about 30 ppm of a compound of the present invention.

In yet further embodiments, the edible composition comprises 0.001 ppm, 0.005 ppm, 0.01 ppm, 0.05 ppm, 0.1 ppm, 0.5 ppm, 1 ppm, 2 ppm, 3 ppm, 4 ppm, 5 ppm, 6 ppm, 7 ppm, 7.5 ppm, 8 ppm, 9 ppm, or 10 ppm of a compound of the present invention. In some embodiments, the edible composition comprises 11 ppm, 12 ppm, 13 ppm, 14 ppm, 15 ppm, 16 ppm, 17 ppm, 18 ppm, 19 ppm, 20 ppm, 21 ppm, 22 ppm, 23 ppm, 24 ppm, 25 ppm, 26 ppm, 27 ppm, 28 ppm, 29 ppm, or 30 ppm of a compound of the present invention.

In still further embodiments, the edible composition comprises about 31 ppm, about 32 ppm, about 33 ppm, about 34 ppm, about 35 ppm, about 36 ppm, about 37 ppm, about 38 ppm, about 39 ppm, about 40 ppm, about 41 ppm, about 42 ppm, about 43 ppm, about 44 ppm, about 45 ppm, about 46 ppm, about 47 ppm, about 48 ppm, about 49 ppm, or about 50 ppm of a compound of the present invention.

In still further embodiments, the edible composition comprises 31 ppm, 32 ppm, 33 ppm, 34 ppm, 35 ppm, 36 ppm, 37 ppm, 38 ppm, 39 ppm, 40 ppm, 41 ppm, 42 ppm, 43 ppm, 44 ppm, 45 ppm, 46 ppm, 47 ppm, 48 ppm, 49 ppm, or 50 ppm of a compound of the present invention.

In some embodiments, the edible composition comprises more than about 0.001 ppm, 0.005 ppm, 0.01 ppm, 0.05 ppm, 0.01 ppm, 0.5 ppm, 1 ppm, 5 ppm, 10 ppm, 15 ppm, 20 ppm, 25 ppm, or 30 ppm of a compound of the present invention, up to, for example, about 30 ppm or 50 ppm. In additional embodiments, the edible composition comprises less than about 50 ppm, 30 ppm, 25 ppm, 20 ppm, 15 ppm, 10 ppm, 5 ppm, 1 ppm, 0.5 ppm, 0.1 ppm, 0.05 ppm, 0.01 ppm, 0.005 ppm, or 0.001 ppm of a compound of the present invention. In yet additional embodiments, the edible composition comprises less than about 30 ppm, 10 ppm, or 1 ppm of a compound of the present invention.

In some embodiments, the edible composition comprises more than 0.001 ppm, 0.005 ppm, 0.01 ppm, 0.05 ppm, 0.01 ppm, 0.5 ppm, 1 ppm, 5 ppm, 10 ppm, 15 ppm, 20 ppm, 25 ppm, or 30 ppm of a compound of the present invention, up to, for example, 30 ppm or 50 ppm. In additional embodiments, the edible composition comprises less than 50 ppm, 30 ppm, 25 ppm, 20 ppm, 15 ppm, 10 ppm, 5 ppm, 1 ppm, 0.5 ppm, 0.1 ppm, 0.05 ppm, 0.01 ppm, 0.005 ppm, or 0.001 ppm of a compound of the present invention. In yet additional embodiments, the edible composition comprises less than 30 ppm, 10 ppm, or 1 ppm of a compound of the present invention.

Further, when the edible composition comprises a natural high-potency sweetener, such as rebaudioside A, the amount of the sweetener added varies depending on the nature of the edible composition, the amount of sweetness required and the presence of other compounds in the composition. Rebaudioside A, for example, may be present at a concentration between about 25-725 ppm, 50-700 ppm, 75-675 ppm, 100-650 ppm, 125-625 ppm, 150-600 ppm, 200-550 ppm, 250-500 ppm, and any concentration between these ranges.

Further, when the edible composition comprises a natural high-potency sweetener, such as rebaudioside A, the amount of the sweetener added varies depending on the nature of the edible composition, the amount of sweetness required and the presence of other compounds in the composition. Rebaudioside A, for example, may be present at a concentration between 25-725 ppm, 50-700 ppm, 75-675 ppm, 100-650 ppm, 125-625 ppm, 150-600 ppm, 200-550 ppm, 250-500 ppm, and any concentration between these ranges.

In general, a compound of the present invention in an edible composition may be present at a concentration between about 0.001 ppb and 1000 ppb. In some embodiments, the edible composition comprises between about 0.005 to 500 ppb; 0.01 to 200 ppb; 0.01 to 150 ppb; 0.01 to 100 ppb; 0.05 to 50 ppb; 0.1 to 5 ppm; 0.1 to 10 ppb; 1 to 10 ppb; 1 to 30 ppb; 1 to 50 ppb; 10 to 30 ppb; 10 to 50 ppb; 30 to 50 ppb; 50 to 100 ppb; 100 to 150 ppb; 150 to 200 ppb; 75 to 100 ppb; 100 to 125 ppb; or 125 to 150 ppb of a compound of the present invention. In yet further embodiments, the edible composition comprises about 0.1 to 30 ppb, 1 to 30 ppb, 1 to 50 ppb, 50 to 100 ppb, 100 to 150 ppb, or 150 to 200 ppb of a compound of the present invention. In additional embodiments, the edible composition comprises about 0.1 to 5 ppb; 0.1 to 4 ppb; 0.1 to 3 ppb; 0.1 to 2 ppb; 0.1 to 1 ppb: 0.5 to 5 ppb; 0.5 to 4 ppb: 0.5 to 3 ppb: 0.5 to 2 ppb: 0.5 to 1.5 ppb: 0.5 to 1 ppb; 5 to 15 ppb; 6 to 14 ppb; 7 to 13 ppb; 8 to 12 ppb; 9 to 11 ppb; 25 to 35 ppb; 26 to 34 ppb; 27 to 33 ppb; 28 to 32 ppb; 29 to 31 ppb; 48 to 52 ppb; 53 to 58 ppb; 59 to 64 ppb; 65 to 70 ppb; 70 to 75 ppb; 75 to 80 ppb; 80 to 85 ppb; 85 to 90 ppb; 90 to 95 ppb; 95 to 100 ppb; 100 to 105 ppb; 105 to 110 ppb; 110 to 115 ppb, 115 to 120 ppb 120 to 125 ppb, 125 to 130 ppb, 130 to 135 ppb; 135 to 140 ppb 140 to 145 ppb; 145 to 150 ppb; 150 to 155 ppb; or 155 to 160 ppb.

In some embodiments, a compound of the present invention in an edible composition may be present at a concentration between 0.001 ppb and 1000 ppb. In some embodiments, the edible composition comprises between 0.005 to 500 ppb; 0.01 to 200 ppb; 0.01 to 150 ppb; 0.01 to 100 ppb; 0.05 to 50 ppb; 0.1 to 5 ppm; 0.1 to 10 ppb; 1 to 10 ppb; 1 to 30 ppb; 1 to 50 ppb; 10 to 30 ppb; 10 to 50 ppb; 30 to 50 ppb; 50 to 100 ppb; 100 to 150 ppb; 150 to 200 ppb; 75 to 100 ppb; 100 to 125 ppb; or 125 to 150 ppb of a compound of the present invention. In yet further embodiments, the edible composition comprises 0.1 to 30 ppb, 1 to 30 ppb, 1 to 50 ppb, 50 to 100 ppb, 100 to 150 ppb, or 150 to 200 ppb of a compound of the present invention. In additional embodiments, the edible composition comprises 0.1 to 5 ppb; 0.1 to 4 ppb; 0.1 to 3 ppb; 0.1 to 2 ppb; 0.1 to 1 ppb; 0.5 to 5 ppb; 0.5 to 4 ppb; 0.5 to 3 ppb; 0.5 to 2 ppb; 0.5 to 1.5 ppb; 0.5 to 1 ppb; 5 to 15 ppb; 6 to 14 ppb; 7 to 13 ppb; 8 to 12 ppb; 9 to 11 ppb; 25 to 35 ppb; 26 to 34 ppb; 27 to 33 ppb; 28 to 32 ppb; 29 to 31 ppb; 48 to 52 ppb; 53 to 58 ppb; 59 to 64 ppb; 65 to 70 ppb; 70 to 75 ppb; 75 to 80 ppb; 80 to 85 ppb; 85 to 90 ppb; 90 to 95 ppb; 95 to 100 ppb; 100 to 105 ppb; 105 to 110 ppb; 110 to 115 ppb; 115 to 120 ppb; 120 to 125 ppb; 125 to 130 ppb; 130 to 135 ppb; 135 to 140 ppb; 140 to 145 ppb; 145 to 150 ppb; 150 to 155 ppb; or 155 to 160 ppb.

In yet further embodiments, the edible composition comprises about 0.1 ppb, about 0.5 ppb, about 1 ppb, about 2 ppb, about 3 ppb, about 4 ppb, about 5 ppb, about 6 ppb, about 7 ppb, about 7.5 ppb, about 8 ppb, about 9 ppb, or about 10 ppb of a compound of the present invention. In some embodiments, the edible composition comprises about 11 ppb, about 12 ppb, about 13 ppb, about 14 ppb, about 15 ppb, about 16 ppb, about 17 ppb, about 18 ppb, about 19 ppb, about 20 ppb, about 21 ppb, about 22 ppb, about 23 ppb, about 24 ppb, about 25 ppb, about 26 ppb, about 27 ppb, about 28 ppb, about 29 ppb, or about 30 ppb of a compound of the present invention.

In yet further embodiments, the edible composition comprises 0.1 ppb, 0.5 ppb, 1 ppb, 2 ppb, 3 ppb, 4 ppb, 5 ppb, 6 ppb, 7 ppb, 7.5 ppb, 8 ppb, 9 ppb, or 10 ppb of a compound of the present invention. In some embodiments, the edible composition comprises 11 ppb, 12 ppb, 13 ppb, 14 ppb, 15 ppb, 16 ppb, 17 ppb, 18 ppb, 19 ppb, 20 ppb, 21 ppb, 22 ppb, 23 ppb, 24 ppb, 25 ppb, 26 ppb, 27 ppb, 28 ppb, 29 ppb, or 30 ppb of a compound of the present invention.

In still further embodiments, the edible composition comprises about 31 ppb, about 32 ppb, about 33 ppb, about 34 ppb, about 35 ppb, about 36 ppb, about 37 ppb, about 38 ppb, about 39 ppb, about 40 ppb, about 41 ppb, about 42 ppb, about 43 ppb, about 44 ppb, about 45 ppb, about 46 ppb, about 47 ppb, about 48 ppb, about 49 ppb, or about 50 ppb of a compound of the present invention.

In still further embodiments, the edible composition comprises 31 ppb, 32 ppb, 33 ppb, 34 ppb, 35 ppb, 36 ppb, 37 ppb, 38 ppb, 39 ppb, 40 ppb, 41 ppb, 42 ppb, 43 ppb, 44 ppb, 45 ppb, 46 ppb, 47 ppb, 48 ppb, 49 ppb, or 50 ppb of a compound of the present invention.

In still further embodiments, the edible composition comprises about 51 ppb, about 52 ppb, about 53 ppb, about 54 ppb, about 55 ppb, about 56 ppb, about 57 ppb, about 58 ppb, about 59 ppb, about 60 ppb, about 61 ppb, about 62 ppb, about 63 ppb, about 64 ppb, about 65 ppb, about 66 ppb, about 67 ppb, about 68 ppb, about 69 ppb, about 70 ppb, about 71 ppb, about 72 ppb, about 73 ppb, about 74 ppb, about 75 ppb, about 76 ppb, about 77 ppb, about 78 ppb, about 79 ppb, about 80 ppb, about 81 ppb, about 82 ppb, about 83 ppb, about 84 ppb, about 85 ppb, about 86 ppb, about 87 ppb, about 88 ppb, about 89 ppb, about 90 ppb, about 91 ppb, about 92 ppb, about 93 ppb, about 94 ppb, about 95 ppb, about 96 ppb, about 97 ppb, about 98 ppb, about 99 ppb, or about 100 ppb of a compound of the present invention.

In still further embodiments, the edible composition comprises 51 ppb, 52 ppb, 53 ppb, 54 ppb, 55 ppb, 56 ppb, 57 ppb, 58 ppb, 59 ppb, 60 ppb, 61 ppb, 62 ppb, 63 ppb, 64 ppb, 65 ppb, 66 ppb, 67 ppb, 68 ppb, 69 ppb, 70 ppb, 71 ppb, 72 ppb, 73 ppb, 74 ppb, 75 ppb, 76 ppb, 77 ppb, 78 ppb, 79 ppb, 80 ppb, 81 ppb, 82 ppb, 83 ppb, 84 ppb, 85 ppb, 86 ppb, 87 ppb, 88 ppb, 89 ppb, 90 ppb, 91 ppb, 92 ppb, 93 ppb, 94 ppb, 95 ppb, 96 ppb, 97 ppb, 98 ppb, 99 ppb, or 100 ppb of a compound of the present invention.

In still further embodiments, the edible composition comprises about 101 ppb, about 102 ppb, about 103 ppb, about 104 ppb, about 105 ppb, about 106 ppb, about 107 ppb, about 108 ppb, about 109 ppb, about 110 ppb, about 111 ppb, about 112 ppb, about 113 ppb, about 114 ppb, about 115 ppb, about 116 ppb, about 117 ppb, about 118 ppb, about 119 ppb, about 120 ppb, about 121 ppb, about 122 ppb, about 123 ppb, about 124 ppb, about 125 ppb, about 126 ppb, about 127 ppb, about 128 ppb, about 129 ppb, about 130 ppb, about 131 ppb, about 132 ppb, about 133 ppb, about 134 ppb, about 135 ppb, about 136 ppb, about 137 ppb, about 138 ppb, about 139 ppb, about 140 ppb, about 141 ppb, about 142 ppb, about 143 ppb, about 144 ppb, about 145 ppb, about 146 ppb, about 147 ppb, about 148 ppb, about 149 ppb, or about 150 ppb of a compound of the present invention.

In still further embodiments, the edible composition comprises 101 ppb, 102 ppb, 103 ppb, 104 ppb, 105 ppb, 106 ppb, 107 ppb, 108 ppb, 109 ppb, 110 ppb, 111 ppb, 112 ppb, 113 ppb, 114 ppb, 115 ppb, 116 ppb, 117 ppb, 118 ppb, 119 ppb, 120 ppb, 121 ppb, 122 ppb, 123 ppb, 124 ppb, 125 ppb, 126 ppb, 127 ppb, 128 ppb, 129 ppb, 130 ppb, 131 ppb, 132 ppb, 133 ppb, 134 ppb, 135 ppb, 136 ppb, 137 ppb, 138 ppb, 139 ppb, 140 ppb, 141 ppb, 142 ppb, 143 ppb, 144 ppb, 145 ppb, 146 ppb, 147 ppb, 148 ppb, 149 ppb, or 150 ppb of a compound of the present invention.

In some embodiments, the edible composition comprises more than about 0.5 ppb, 1 ppb, 5 ppb, 10 ppb, 15 ppb, 20 ppb, 25 ppb, or 30 ppb of a compound of the present invention, up to, for example, about 30 ppb or 50 ppb. In additional embodiments, the edible composition comprises less than about 50 ppb, 30 ppb, 25 ppb, 20 ppb, 15 ppb, 10 ppb, 5 ppb, 1 ppb, or 0.5 ppb of a compound of the present invention. In some embodiments, the edible composition comprises more than about 50 ppb, 55 ppb, 60 ppb, 65 ppb, 70 ppb, 75 ppb, 80 ppb, 90 ppb, 95 ppb, 100 ppb, 105 ppb, 110 ppb, 115 ppb, 120 ppb, 125 ppb, 130 ppb, 135 ppb, 140 ppb, 145 ppb, or 150 ppb of a compound of the present invention, up to, for example, about 100 ppb or 150 ppb. In additional embodiments, the edible composition comprises less than about 150 ppb, 125 ppb, 100 ppb, 75 ppb, or 50 ppb of a compound of the present invention. In yet additional embodiments, the edible composition comprises less than about 150 ppb, 100 ppb, 75 ppb, or 50 ppb of a compound of the present invention.

In some embodiments, the edible composition comprises more than 0.5 ppb, 1 ppb, 5 ppb, 10 ppb, 15 ppb, 20 ppb, 25 ppb, or 30 ppb of a compound of the present invention, up to, for example, 30 ppb or 50 ppb. In additional embodiments, the edible composition comprises less than 50 ppb, 30 ppb, 25 ppb, 20 pp, 15 ppb, 10 ppb, 5 ppb, 1 ppb, or 0.5 ppb of a compound of the present invention. In some embodiments, the edible composition comprises more than 50 ppb, 55 ppb, 60 ppb, 65 ppb, 70 ppb, 75 ppb, 80 ppb, 90 ppb, 95 ppb, 100 ppb, 105 ppb, 110 ppb, 115 ppb, 120 ppb, 125 ppb, 130 ppb, 135 ppb, 140 ppb, 145 ppb, or 150 ppb of a compound of the present invention, up to, for example, 100 ppb or 150 ppb. In additional embodiments, the edible composition comprises less than 150 ppb, 125 ppb, 100 ppb, 75 ppb, or 50 ppb of a compound of the present invention. In yet additional embodiments, the edible composition comprises less than 150 ppb, 100 ppb, 75 ppb, or 50 ppb of a compound of the present invention.

Further, when the edible composition comprises a natural high-potency sweetener, such as rebaudioside A, the amount of the sweetener added varies depending on the nature of the edible composition, the amount of sweetness required and the presence of other compounds in the composition. Rebaudioside A, for example, may be present at a concentration between about 25-725 ppb, 50-700 ppb, 75-675 ppb, 100-650 ppb, 125-625 ppb, 150-600 ppb, 200-550 ppb, 250-500 ppb, and any concentration between these ranges.

Further, when the edible composition comprises a natural high-potency sweetener, such as rebaudioside A, the amount of the sweetener added varies depending on the nature of the edible composition, the amount of sweetness required and the presence of other compounds in the composition. Rebaudioside A, for example, may be present at a concentration between 25-725 ppb, 50-700 ppb, 75-675 ppb, 100-650 ppb, 125-625 ppb, 150-600 ppb, 200-550 ppb, 250-500 ppb, and any concentration between these ranges.

In some embodiments, a high potency sweetener or artificial sweetener is added to the edible composition in an amount sufficient to replace sugar. In some embodiments, the artificial sweetener or high potency sweetener has a bitter taste or aftertaste. In some embodiments, the high potency sweetener is rebaudioside A. For example, the amount of rebaudioside A in the edible composition may range from about 0.001 to about 0.01 times the replaced sugar depending upon the application, e.g., if about 100 mg of sugar is replaced, about 0.1 to about 1 mg of rebaudioside A is added. Typically, rebaudioside A will be added in about 0.005 times the amount of sugar being replaced. In some embodiments, the amount of rebaudioside A in the edible composition may range from 0.001 to about 0.01 times the replaced sugar depending upon the application, e.g., if 100 mg of sugar is replaced, 0.1 to 1 mg of rebaudioside A is added. In some embodiments, rebaudioside A will be added in 0.005 times the amount of sugar being replaced.

In some embodiments, the package contains an edible composition comprising a compound of the present invention and a bitter tastant. In some embodiments, the package contains an edible composition comprising a compound of the present invention and bitter tasting sweetener.

In some embodiments, the edible compositions of the present invention are compositions suitable to be used as seasonings, as ingredients in food products or as condiments. In such embodiments, the edible composition may or may not contain a bitter tastant. For example, the edible composition may be used in, e.g., a seasoning which comprises a bitter tastant such as, e.g., cyclamate, rebaudioside A, acesulfame K, saccharin, stevioside, NHDC, or advantame. Such seasonings can be used in the place of table sugar (i.e., sucrose) to season prepared food products. Alternatively, the edible composition may be used in, e.g., a seasoning which does not contain a bitter tastant. Such seasonings can be used to season prepared food products which contain a bitter tastant (either inherently present or added during preparation) in order to reduce the bitter taste associated with the bitter tastant. In some embodiments, the edible composition is a seasoning comprising rebaudioside A and a compound of the invention. When the edible composition of the invention is used as a seasoning, the compound of the invention may be present in an amount such that it can be added to another composition to achieve the concentrations disclosed, supra.

Alternatively, the edible compositions may be used for medicinal or hygienic purposes, for example, in soaps, shampoos, mouthwash, medicines, pharmaceuticals, cough syrup, nasal sprays, toothpaste, dental adhesives, tooth whiteners, glues (e.g., on stamps and envelopes), and toxins used in insect and rodent control.

### Food Product

In some embodiments, the edible composition is a food product. According to such embodiments, the food product comprises (a) a food stuff; and (b) a compound of Formula (I), or a comestibly or biologically acceptable salt, derivative, enantiomer, or diastereomer thereof, as described herein or Compound 1, or a comestibly or biologically acceptable salt, derivative, or enantiomer thereof, as described herein, or combinations of any of the aforementioned.

In some embodiments, the food product further comprises a bitter tastant, as described herein. In some embodiments, the bitter tastant is rebaudioside A or stevioside, preferably rebaudioside A.

In some embodiments, the food product further comprises one or more additional flavor modifiers. In some embodiments, the food product further comprises one or more sweet taste improving compositions or sweet taste improving additives.

In some embodiments, the food product further comprises one or more additional components selected from the group consisting of preservatives, nutritives, flavorants or additional flavor modifiers, which may lack an inherent flavor.

In some embodiments the food product is a beverage.

### Pharmaceutical Composition

In some embodiments, the edible composition is a pharmaceutical composition. According to such embodiments, the pharmaceutical composition comprises (a) a bitter tasting pharmaceutically active ingredient; and (b) a compound of Formula (I), or a comestibly or biologically acceptable salt, derivative, enantiomer, or diastereomer thereof, as described herein or Compound 1, or a comestibly or biologically acceptable salt, derivative, or enantiomer thereof, as described herein, or combinations of any of the aforementioned.

According to some embodiments, the pharmaceutical composition can comprise any bitter tasting pharmaceutically active ingredient. Non-limiting examples of bitter pharmaceutical compounds include: acetaminophen, ampicillin, azithromycin, chlorpheniramine, cimetidine, dextromethorphan, diphenhydramine, erythromycin, esomeprazole, guaifenesin, ibuprofen, penicillin, phenylbutazone, pseudoephedrine, ranitidine, sildenafil, spironolactone, statins (including, but not limited to, atorvastatin, cerivastatin, fluvastatin, louvastatin, mevastatin, pitavastatin, pravastatin, rosuvastatin, and simvastatin) and theophylline.

In some embodiments, the invention provides a pharmaceutical composition comprising (a) a pharmaceutically active ingredient; (b) a compound of Formula (I), or a comestibly or biologically acceptable salt, derivative, enantiomer, or diastereomer thereof, as described herein or Compound 1, or a comestibly or biologically acceptable salt, derivative, or enantiomer thereof, as described herein, or combinations of any of the aforementioned; and (c) a bitter tastant. In such embodiments, the pharmaceutical compositions may comprise any pharmaceutically active ingredient.

In some embodiments, the pharmaceutical composition further comprises one or more additional flavor modifiers. In some embodiments, the pharmaceutical composition further comprises one or more sweet taste improving compositions or sweet taste improving additives.

In some embodiments, the pharmaceutical composition further comprises one or more additional components selected from the group consisting of preservatives, nutritives, flavorants or flavor modifiers, which may lack an inherent flavor.

### Consumer Product

In some embodiments, the edible compositions is a consumer product. According to such embodiments, the consumer product comprises (a) a bitter tastant; and (b) a compound of Formula I, or a comestibly or biologically acceptable salt, derivative, enantiomer, or diastereomer thereof, as described herein or Compound 1, or a comestibly or biologically acceptable salt, derivative, or enantiomer thereof as described herein, or combinations of any of the aforementioned.

In another embodiment, the invention provides a consumer product comprising (a) high potency sweetener; and (b) a compound of Formula (I), or a comestibly or biologically acceptable salt, derivative, enantiomer, or diastereomer thereof as described herein, or Compound 1, or a comestibly or biologically acceptable salt, derivative, or enantiomer thereof, as described herein, or combinations of any of the aforementioned. In some embodiments, the high potency sweetener is rebaudioside A or stevioside, preferably rebaudioside A.

In some embodiments, the invention provides a consumer product for reducing bitter taste of a bitter tastant, wherein said consumer product comprises a compound of Formula (I), or a comestibly or biologically acceptable salt, derivative, enantiomer, or diastereomer thereof, as described herein, or Compound 1, or a comestibly or biologically acceptable salt, derivative, or enantiomer thereof, as described herein, or combinations of any of the aforementioned. In some embodiments, the bitter tastant is rebaudioside A or stevioside, preferably rebaudioside A.

In some embodiments, the consumer product further comprises one or more additional flavor modifiers. In some embodiments, the consumer product further comprises one or more sweet taste improving compositions or sweet taste improving additives.

In some embodiments, the consumer product further comprises one or more additional components selected from the group consisting of preservatives, nutritives, flavorants or additional flavor modifiers, which may lack an inherent flavor.

### Method of Preparing an Edible Composition

According to another aspect, the invention provides a method of preparing an edible composition. The method comprises: (a) providing a comestibly acceptable carrier; and (b) adding to the comestibly acceptable carrier a compound of Formula (I), or a comestibly or biologically acceptable salt, derivative, enantiomer, or diastereomer thereof, as described herein, or Compound 1, or a comestibly or biologically acceptable salt, derivative, or enantiomer thereof, as described herein, or combinations of any of the aforementioned, with the comestibly acceptable carrier. In some embodiments, the compound of the invention has been dissolved in a solvent prior to the addition step (b).

In some embodiments, the comestibly acceptable carrier is inherently bitter. In such embodiments, the comestibly acceptable carrier may inherently contain a bitter tastant. In some embodiments, the inherent bitter tastant is a high potency sweetener.

In some embodiments, the method of preparing an edible composition further comprises: (c) adding a bitter tastant. In some embodiments, the bitter tastant is a high potency sweetener. In some embodiments, the high potency sweetener is rebaudioside A or stevioside, preferably rebaudioside A. In some embodiments, the bitter tastant is added before the compound of the present invention. In some embodiments, the bitter tastant is added after the compound of the present invention. In some embodiments, the compounds of the present invention are combined with the bitter tastant and then combined with the comestibly acceptable carrier. In some embodiments, the compound of the present invention is combined sequentially with the comestibly acceptable carrier and then the bitter tastant. In yet further embodiments, the compounds of the present invention are combined with a mixture of the bitter tastant and the comestibly acceptable carrier.

In some embodiments, a compound of the invention and the bitter tastant, if present, are mixed with the comestibly acceptable carrier. In some embodiments, the compound and the bitter tastant, if present, are sprayed onto or coat the comestibly acceptable carrier. In some embodiments, the compound of the invention is plated on a carbohydrate or salt, encapsulated on a salt or a carbohydrate (spray dried), or co-crystallized with a salt to create a “topping” salt.

In some embodiments, the methods of preparing an edible composition further comprise adding one or more additional components selected from the group consisting of preservatives, nutritives, flavorants or flavor modifiers, which may lack an inherent flavor. In some embodiments, the methods of preparing an edible composition further comprise adding one or more additional flavor modifiers. In some embodiments, the methods of preparing an edible composition further comprises adding one or more sweet taste improving compositions or sweet taste improving additives.

In some embodiments, the edible composition is a consumer product.

### Method of Preparing a Food Product

According to another aspect, the invention provides a method of preparing an edible composition, wherein the edible composition is a food product. The method comprises: (a) providing a foodstuff; and (b) adding to the foodstuff a compound of Formula (I), or a comestibly or biologically acceptable salt, derivative, enantiomer, or diastereomer thereof, as described herein, or Compound 1, or a comestibly or biologically acceptable salt, derivative, or enantiomer thereof, as described herein, or combinations of any of the aforementioned. In some embodiments, the compound of the invention is added in the form of an edible composition comprising the compound of the invention.

In some embodiments, the foodstuff is inherently bitter. In such embodiments, the foodstuff may inherently contain a bitter tastant.

In some embodiments, the method comprises: (a) providing a ibod product; and (b) adding to the food product a compound of Formula (I), or a comestibly or biologically acceptable salt, derivative, enantiomer, or diastereomer thereof, as described herein, or Compound 1, or a comestibly or biologically acceptable salt, derivative, or enantiomer thereof, as described herein, or combinations of any of the aforementioned. In some embodiments, the compound of the invention is added in the form of an edible composition comprising the compound of the invention.

In some embodiments, the food product comprises a bitter tastant. In some embodiments, the bitter tastant is a high potency sweetener. In some embodiments, the high potency sweetener is rebaudioside A or stevioside, preferably rebaudioside A.

In some embodiments, the method of preparing a food product further comprises: (c) adding a bitter tastant. In some embodiments, the bitter tastant is added before the compound of the present invention. In some embodiments, the bitter tastant is added after the compound of the present invention. In some embodiments, the compound of the invention is added with the bitter tastant. In some embodiments, the compound of the present invention is combined with the bitter tastant and then combined with the foodstuff or food product. In some embodiments, the compound of the present invention is combined sequentially with the foodstuff or food product and then the bitter tastant. In yet further embodiments, the compound of the present invention is combined with a mixture of the bitter tastant and the foodstuff or food product.

In some embodiments, the compound and the bitter tastant, if present, are mixed with the foodstuff. In some embodiments, the compound and the bitter tastant, if present, are sprayed onto or coat the foodstuff. In some embodiments, the compound of the invention is plated on a carbohydrate or salt, encapsulated on a salt or a carbohydrate (spray dried), or co-crystallized with a salt to create a “topping” salt.

In further embodiments, the food product further comprises sugar.

In some embodiments, the methods of preparing a food product further comprise adding one or more additional components selected from the group consisting of preservatives, nutritives, flavorants or flavor modifiers, which may lack an inherent flavor. In some embodiments, the methods of preparing a food product further comprises adding one or more sweet taste improving compositions or sweet taste improving additives.

### Method of Preparing a Pharmaceutical Composition

According to another aspect, the invention provides a method of preparing an edible composition, wherein the edible composition is a pharmaceutical composition. The method comprises: (a) providing a pharmaceutically active ingredient; and (b) adding to the pharmaceutically active ingredient a compound of Formula (I), or a comestibly or biologically acceptable salt, derivative, enantiomer, or diastereomer thereof, as described herein, or Compound 1, or a comestibly or biologically acceptable salt, derivative, or enantiomer thereof, as described herein, or combinations of any of the aforementioned, with the pharmaceutically active ingredient. In some embodiments, the compound of the invention is added in the form of an edible composition comprising the compound of the invention.

In some embodiments, the pharmaceutically active ingredient in (a) is inherently bitter. In such embodiments, the pharmaceutically active ingredient may inherently contain a bitter tastant.

In some embodiments, the method of preparing a pharmaceutical composition further comprises: (c) adding a bitter tastant. In some embodiments, the bitter tastant is added before the compound of the present invention. In some embodiments, the bitter tastant is added after the compound of the present invention. In some embodiments, the bitter tastant is added with the compound of the invention. In some embodiments, the compound of the present invention is combined with the bitter tastant and then combined with the pharmaceutically active ingredient. In some embodiments, the compound of the present invention is combined sequentially with the pharmaceutically active ingredient and then the bitter tastant. In yet further embodiments, the compound of the present invention is combined with a mixture of the bitter tastant and the pharmaceutically active ingredient.

In some embodiments, the compound and the bitter tastant, if present, are mixed with the pharmaceutically active ingredient. In some embodiments, the compound and the bitter tastant, if present, are sprayed onto or coat the pharmaceutical composition. In some embodiments, the compound of the invention is encapsulated with the pharmaceutically active ingredient. In some embodiments, the compound of the invention is in a form such that the rate of release is regulated vis a vis the rate of release of the bitter tastant, which in some embodiments is the pharmaceutically active ingredient.

In further embodiments, the pharmaceutical composition further comprises sugar.

In some embodiments, the pharmaceutical composition further comprises a pharmaceutically acceptable carrier. Pharmaceutically acceptable carriers that may be used in these compositions include, but are not limited to, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In some embodiments, the methods of preparing a pharmaceutical composition further comprise adding one or more additional components selected from the group consisting of preservatives, nutritives, flavorants or flavor modifiers, which may lack an inherent flavor. In some embodiments, the methods of preparing a pharmaceutical composition further comprises adding one or more sweet taste improving compositions or sweet taste improving additives.

### Method of Reducing the Amount of Sugar in an Edible Composition or Food Product

According to another embodiment, the invention provides a method of reducing the amount of sugar in an edible composition. In some embodiments, the method comprises: (a) replacing an amount of sugar used in preparing an edible composition with an amount of high potency sweetener, and (b) incorporating into the edible composition an effective amount of a compound of Formula (I), or a comestibly or biologically acceptable salt, derivative, enantiomer, or diastereomer thereof, as described herein, or Compound 1, or a comestibly or biologically acceptable salt, derivative, or enantiomer thereof, as described herein, or combinations of any of the aforementioned.

In some embodiments, the edible composition is a food product. In some embodiments, the edible composition is a pharmaceutical composition. In some embodiments, the edible composition is a consumer product.

In some embodiments, the high potency sweetener is added to the edible composition prior to addition of an effective amount of a compound of the invention. In some embodiments, the high potency sweetener is added to the edible composition subsequent to addition of an effective amount of a compound of the invention. In some embodiments, the high potency sweetener is added to the edible composition concurrent with addition of an effective amount of a compound of the invention. In some embodiments the high potency sweetener is rebaudioside A or stevioside, preferably rebaudioside A.

In some embodiments, the amount of sugar replaced by high potency sweetener is up to 1%, 2%, 3%, 4%, 5%, 6%, 7%, 8%, 9%, 10%, 15%, 20%, 25%, 30%, 35%, 40%, 45%, 50%, 55%, 60%, 65%, 70% 75%, 80%, 85%, 90%, 95% or 100%. These amounts are not meant to be limiting, and increments between the recited percentages are specifically envisioned as part of the invention.

In some embodiments, the amount of compound added reduces the perception of bitter taste in the subject. The bitter taste is completely reduced or partially reduced. In some embodiments, the perception or sweet taste is maintained.

In some embodiments, the amount of compound added is sufficient to permit replacement of up to 1%, 2%, 3%, 4%, 5%, 6%, 7%, 8%, 9%, 10%, 15%, 20%, 25%, 30%, 35%, 40%, 45%, 50%, 55%, 60%, 65%, 70% 75%, 80%, 85%, 90%, 95% or 100% of the amount of sugar present in the edible composition with high potency sweetener. These amounts are not meant to be limiting, and increments between the recited percentages are specifically envisioned as part of the invention. In some embodiments, the amount of compound added is sufficient to permit replacement of up to 25% of the amount of sugar present in the edible composition with high potency sweetener (e.g., rebaudioside A). In some embodiments, the amount of compound added in step (b) is sufficient to permit replacement of up to 50% of the amount of sugar present in the edible composition with high potency sweetener (e.g., rebaudioside A). In some embodiments, the amount of compound added is sufficient to permit replacement of up to 75% of the amount of sugar present in the edible composition with high potency sweetener (e.g., rebaudioside A). In yet further embodiments, the amount of compound added is sufficient to permit replacement of up to 100% of the amount of sugar present in the edible composition with high potency sweetener (e.g., rebaudioside A).

In some embodiments, the method of reducing the amount of sugar in an edible composition comprises maintaining a sweet flavor.

In some embodiments, the method of reducing the amount of sugar in an edible composition or food product further comprises adding one or more additional components selected from the group consisting of preservatives, nutritives, flavorants or flavor modifiers (including sweet taste modifying compositions), which may lack an inherent flavor. In some embodiments, the method of reducing the amount of sugar in an edible composition or food product further comprises adding one or more sweet taste improving compositions or sweet taste improving additives.

### Preparation of the Compounds of the Invention

In some embodiments, the compound of Formula (I) is commercially available, for example from commercial sources such as Sigma-Aldrich® of St. Louis, Mo., USA; TCI America, Portland, Oreg., USA; and ChromaDex®, Irvine, Calif., USA, Indofine of Hillsborough, N.J., USA, Fluka; among others. In some embodiments, it may be natural (e.g., extracted from biomass (for example Abrus cantoniensis, Erythrina caffra) or synthetic. In some embodiments, biosynthetic pathways are used to make the compounds of Formula (I).

### EXAMPLES

In order that this invention be more fully understood, the following examples are set forth. These examples are for the purpose of illustration only and are not to be construed as limiting the scope of the invention in any way.

The test compounds used in the following examples may be obtained from commercial vendors for synthetic and natural compounds, such as Sigma-Aldrich® (Product 434248), TCI (Product A1489), ChromaDex® (Product ASB-00001006) and Indofine (Product 526-31-8).

The taste test panelists used in the following examples were screened based upon and selected for their ability to perceive the bitter taste associated with rebaudioside A. Only panelists capable of perceiving bitter taste participated in the following taste tests.

Due to the complex nature of taste perception in subjects and the inherently subjective nature of the following experiments, individual taste test trials may yield different results for a given compound. The data presented in the following Examples are illustrative of the taste testing results observed.

### Example 1: Determination of Bitterness Reduction Using a Trained Descriptive Analysis (DA) Panel

### Panel A

The effect of the test compounds on the perception of the bitter taste in aqueous and acidified matrices was evaluated using a descriptive analysis methodology with a group of trained panelists as follows.

Candidate panelists were recruited, with prescreening and personal interviews, and were assessed for their ability to detect, recognize and differentiate basic taste attributes or mixtures thereof as part of a standardized acuity test. These candidate panelists were also assessed for their innate ability to identify flavors, and to rank on intensity scales. Other senses such as smell and vision were also included as part of the assessment. Candidates also were screened in their ability to use the language to describe and articulate ideas. Selected candidates proceeded to training as a group in three phases; (1) lexicon development, (2) concept alignment, and (3) scaling descriptors. During lexicon development, panelists evaluate products appropriate for use in the study to generate and align on terms describing the flavor, taste, aromatic, trigeminal, and temporal attributes. During concept alignment, the panel evaluates the products mentioned above to clarify and confirm the attributes that were generated during lexicon development using references standards that appropriately define each attribute, either physical references (e.g., sucrose solutions) or verbal descriptions (e.g., overall flavor). Product terms and concepts are validated during this portion of the study. In the last phase, scaling descriptors the panel participates in a series of exercises focused on ordering and ranking samples according to relative attribute intensity, measuring attribute intensity using a defined length of line scale. Whenever possible different levels or concentration of the references are used as anchors to facilitate the use of the scale. Panelists are provided with blinded references at this stage to evaluate their understanding and perception of the scale.

The panel used in this study was trained to reference bitter taste using caffeine references allocated along the 15 cm scale. The panel was trained using a hybrid approach between descriptive analysis methods (e.g., quantitative descriptive analysis, the sensory spectrum, etc) well-known to those skilled in the art. In order to quantify the bitterness of high potency sweeteners (HPB), this panel was also trained to quantify the difference using a set of stevioside references allocated along the 15 cm scale. The definitions, attributes are references used for this study are outlined in Table 1.

Panel performance was measured at regular intervals using model beverages matrices, with a cohort of 15 panelists demonstrated good performance for reproducibility (measured as a panel average of 79%, and defined as the number of attributes with reproducibility comparable to group performance (p<0.05)), discrimination (measured as a panel average of 97%, and defined as the number of attributes significantly different at a 90% confidence level individually (p<0.1)), and agreement (measured with a cohort average of 90% and defined as the number of attributes that correlate well to the panel consensus (R>0.7)).

A total of 10-15 panelists evaluated the major sensory characteristics of the compounds in aqueous and acidified environment including: sweet, bitter, sour, astringency, HPB, licorice, sweet onset, sweet aftertaste, sweet linger, and bitter aftertaste. The panel was presented with the control and variant samples at ambient temperature, with matrix randomization blocks, and all samples were presented to the panelists blinded and coded using 3-digit random numbers. Samples were presented in monadic order. Panelists used a sip and expectorate procedure for all samples. After each sample assessment, panelists performed a standard palate cleansing protocol, and observed an inter-sample interval time (ISI).

Data was collected and exported electronically utilizing FIZZ sensory software. Data analysis was conducted using SENPAQ version 5.0 software that uses tools such as ANOVA. Fisher's LSD, correlation to determine panel performance as well as significant differences between samples and attributes. Illustrative results are presented in Table 2 and Table 3 and the statistical significance of the numerical values is indicated by differing alphabetical letters. Statistically significant values are indicated by “b” and “c”. Not statistically significant values are indicated by “a”. The effect of Compound 1 on sensory perception in comparison to control samples was statistically ascertained at a 95% significance level using Fisher's LSD tests (Tables 2 and 3). Samples for the first descriptive analysis taste test (Table 2) were prepared by dissolving 5 mg/mL of Compound 1 in ethanol which was subsequently diluted in water to achieve a final solution concentration of 7.5 ppm along with 500 ppm rebaudioside A. Control samples were an aqueous solutions of rebaudioside A at 500 ppm. Samples for the second descriptive analysis taste test (Table 3) were prepared by dissolving 3.75 mg/mL of Compound 1 in ethanol which was subsequently diluted in Orange VitaminWater™ (with RebA concentration of approximately 600 ppm), to achieve a final solution concentration of 5 ppm. Control samples were Orange VitaminWater™ which contains Stevia Leaf Extract (rebaudioside A).

TABLE 1
Definitions and references for each attribute evaluated in this study.
Bitter Blocker Lexicon

| Category | Attribute | Definition | Reference | Intensity | | |
|---|---|---|---|---|---|---|
| Taste | Sweet | Basic taste sensation | Sucrose in water | 1 | 1% | |
| | | associated w/sucrose | | 5 | 5% | |
| | | | | 10 | 10% | |
| | | | | 15 | 15% | |
| | Bitter | Basic taste sensation | Caffeine in water | 0 | 0% | |
| | | associated w/caffeine | | 5 | 0.08% | |
| | | | | 10 | 0.15% | |
| | | | | 15 | 0.20% | |
| | HPB | Basic bitter taste sensation | Stevioside in water | 0 | 0 | ppm |
| | | associated w/high potency | | 2.5 | 50 | ppm |
| | | sweeteners | | 6 | 250 | ppm |
| | | | | 9 | 500 | ppm |
| | | | | 12 | 750 | ppm |
| | | | | 15 | 1000 | ppm |
| | Sour | Basic taste sensation | Citric Acid in water | 0 | 0% | |
| | | associated w/Citric Acid | | 5 | 0.08% | |
| | | | | 10 | 0.15% | |
| | | | | 15 | 0.20% | |
| Aroma/Flavor | Licorice | Flavor associated with licorice | 1 fennel seed tea | 0 | 0% | |
| | | root | bag in 1 L of water | 5 | 9% | |
| | | | for 10 min. | 10 | 17.5% | |
| | | | | 15 | 25% | |
| Mouthfeel | Astringent | Shrinking, puckering or drying | K Alum in water | 0 | 0% | |
| | | of oral cavity caused by | | 5 | 0.01% | |
| | | substances such as tannic acid | | 10 | 0.02% | |
| | | or alums | | 15 | 0.03% | |
| Onset | Sweet | Time rated to reach maximum | Sucrose | 3 | 60 | g/L |
| | Appearance | sweet intensity | Stevia | 8 | 0.63 | g/L |
| | Time | | Monoammonium | 14 | 4.9 | g/L |

| | | | | |
|---|---|---|---|---|
| | | Glycyrrhizinate | | |
| Sweet | Amount of sweetness that | Sucrose in water | 1 | 1% |
| aftertaste | remains in the mouth 1 | | 5 | 5% |
| | minute after expectoration | | 10 | 10% |
| | | | 15 | 15% |
| Bitter | Amount of bitterness that | Caffeine in water | 0 | 0% |
| aftertaste | remains in the mouth 1 | | 5 | 0.08% |
| | minute after expectoration | | 10 | 0.15% |
| | | | 15 | 0.20% |
| Sweet Linger | Amount of sweetness that | Sucrose in water | 1 | 1% |
| | remains in the mouth 3 | | 5 | 5% |
| | minute after expectoration | | 10 | 10% |
| | | | 15 | 15% |

TABLE 2
Results of Taste Testing with Compound
1, Aqueous (Descriptive Analysis)

| | | | 500 ppm RebA + | |
|---|---|---|---|---|
| | 500 ppm RebA | | Compound 1 @7.5 ppm | |
| Bitter | 5.86 | a | 5.32 | b |
| HPB | 8.41 | a | 7.70 | b |
| Licorice | 7.43 | a | 6.58 | b |
| Sweet Aft | 5.25 | a | 4.71 | b |
| Bitter Aft | 3.77 | a | 3.18 | b |
| Sweet Linger | 3.11 | a | 2.55 | b |

TABLE 3
Results of Taste Testing with Compound
1, Orange VitaminWater ™ (Descriptive
Analysis)

| | Orange Vitamin | | Orange Vitamin Water Zero + | |
|---|---|---|---|---|
| | Water Zero | | Compound 1 @5 ppm | |
| Bitter | 5.60 | a | 4.97 | b |
| HPB | 7.68 | a | 6.83 | c |
| Licorice | 3.00 | a | 2.74 | b |
| Swt Aft | 4.46 | a | 3.89 | b |
| Bit Aft | 3.52 | a | 2.94 | b |
| Swt Linger | 2.48 | a | 1.99 | b |

It was later observed that not all of Compound 1 solubilized. Accordingly, the actual concentrations of Compound 1 tested were slightly below the concentrations reported above.

### Panel B

Fully solubilized Compound 1 was analyzed by a second panel, who was trained as described for Panel A, supra. Full solubilization of Compound 1 was achieved using a 1:1 ratio of 200 proof ethanol to water as a solvent to prepare stock solutions. The fully solubilized samples were diluted in the test matrix for a final ethanol concentration of 0.2%. These samples were subjected to first and second descriptive analysis taste tests as described for Panel A, supra. Illustrative results are presented in Table 4 and Table 5 and the statistical significance of the numerical values is indicated by differing alphabetical letters. Statistically significant values are indicated by “b” and “c”. Not statistically significant values are indicated by “a”.

TABLE 4
Results of Taste Testing with Compound
1, Aqueous (Descriptive Analysis)

| | | | 500 ppm Reb-A + | |
|---|---|---|---|---|
| | 500 ppm Reb-A | | Compound 1 @0.3 ppm | |
| Bitter | 3.8 | a | 3.4 | b |
| HPB | 5.7 | a | 5 | b |
| Bitter Aft | 1.9 | a | 1.6 | b |

TABLE 5
Results of Taste Testing with Compound
1, Orange VitaminWater ™ (Descriptive
Analysis)

| | | | 500 ppm Reb-A + | |
|---|---|---|---|---|
| | 500 ppm Reb-A | | Compound1 @0.25 ppm | |
| Bitter | 4.4 | a | 3.9 | b |
| HPB | 6.2 | a | 5.4 | b |

### Example 2: Effect of the Bitter Blocker Candidates in Humans (Sensory Panelists) Using a Discrimination Testing with Sureness Test and R-Index Analysis

The discrimination testing with sureness extends the simple yes/no task of discrimination test in a signal detection procedure to include a sureness rating scale. The discrimination test principle is based on two alternative forced choice (2-AFC) manner, in this methodology, panelists are presented with multiple samples, in which one sample is a control, and the other sampled are test variants. A blind control is included in the test variants along with a known control. Panelists are asked to first discriminate between the known control and a variant by identifying which the sample is less bitter. Second, panelists rate their sureness of their response (e.g., sure, unsure, or guess).

To minimize bias and prevent panelist expectation of sample order, a Latin square randomization design is utilized. The samples within a set will be randomized and the sets are randomized via Latin square. The number of presentations is equally distributed. Each of the test samples is evaluated against a negative control sample to calculate R-index score and associated p-value. P-value is utilized to determine if the two samples are significantly different from each other. R-index is utilized to indicate the degree of difference between the test and the control sample. R-index scores range from 50%-100%, where higher the R-index, larger is the difference in “bitterness” between the two samples.

The blocker candidates were evaluated in a “sip and spit” format using this methodology and analysis and were the results of at least 30 observations (e.g., 15 panelists and 2 replications or 10 panelists 3 replications). Illustrative results are presented in Table 6.

TABLE 6
RebA Latin Square-2AFC Aqueous

| | Discrim | | | | | |
|---|---|---|---|---|---|---|
| Sweetener | or DA | Matrix | ppm | N | R-index | p-value |
| Compound | Discrim. | Aqueous | 5 | 34 | 63.3 | 0.05 |
| 1 in 500 ppm | Sureness | | | | | |
| RebA | | | | | | |

### Example 3: Effect of the Bitter Blocker Candidates on the Perception of the Bitter Taste in Grape-Flavored Cough Syrup in Humans (Sensory Panelists)

Compound 1 was screened for a reduction of bitter taste due to a bitter tastant in children's grape flavored cough syrup (Robitussin Cough and Cold CF™, Lot #: R10494, Exp. 07/15). Compound 1 was solubilized in polyethylene glycol (PG), dosed into the cough syrup, and placed in 3 mL amounts in sample vials. Control samples contained an equal amount of PG to samples containing Compound 1. Testers (n=7) were asked to use a plastic transfer pipette for tasting, rinsing with water between samples and choosing the less bitter sample via the two alternative forced choice (2-AFC) method. All samples were “sip and spit.” Illustrative results are presented in Table 7.

TABLE 7
Cough Syrup 2-AFC Analysis

| | | Compound | Control | |
|---|---|---|---|---|
| | | Chosen as | Chosen as | No Choice |
| Compound | Dose (ppm) | Less Bitter | Less Bitter | Made |
| Compound 1 | 0.1 | 5 | 1 | 1 |

### Example 4: Effect of the Bitter Blocker Candidates at 50 ppb or 100 ppb on the Perception of the Bitter Taste in Grape-Flavored Cough Syrup in Humans (Sensory Panelists)

Compound 1 was screened for a reduction of bitter taste due to a bitter tastant in children's grape flavored cough syrup (Robitussin Cough and Cold CF™, Lot #: R10494, Exp. 07/15). Compound 1 was solubilized in polyethylene glycol (PG), dosed into the cough syrup, and placed in 2.5 mL amounts in sample vials to afford 50 ppb or 100 ppb Compound 1 in the samples. Control samples contained an equal amount of PG to samples containing Compound 1. Testers (n=11) were asked to use a plastic transfer pipette for tasting, rinsing with water between samples and choosing the less bitter sample via the two alternative forced choice (2-AFC) method. All samples were “sip and spit” Illustrative results are presented in Table 8.

TABLE 8
Cough Syrup 2-AFC Analysis with
50 ppb and 100 ppb Compound 1

| | Cmpd. | Chosen | Chosen | Chosen | Chosen |
|---|---|---|---|---|---|
| Compound/ | Chosen | in 1^{st} | in 2^{nd} | if 1^{st} | if 2^{nd} |
| use level | Overall | Pair | Pair | in Pair | in Pair |
| Compound 1 @ | 7/11 | 5/6 | 2/5 | 5/6 | 2/5 |
| 50 ppb | | | | | |
| Compound 1 @ | 7/11 | 3/5 | 4/6 | 4/5 | 3/6 |
| 100 ppb | | | | | |

### Example 5: Screening for the Inherent Sweetness of Compound 1

### Preparation of Samples for Sensory Taste Tests:

Sucrose solutions were prepared by adding sucrose to water to achieve the desired concentrations. Compounds were first prepared as 250-fold concentrated stocks in a 50% ethanol (200 proof), 50% water solution. These concentrated stocks were then diluted in water to achieve a final ethanol concentration of 0.2%. The control solutions were also normalized to 0.2% ethanol. This level of ethanol has previously been shown to not contribute any perceived sweetness.

### Sensory Methodology: Inherent Sweetness Assessment Using 2-AFC Method:

Compound 1 was evaluated in an aqueous solution for sweetness perception in a 2-AFC test. This test was a double-blinded, randomized study where taste panelists evaluate a pair of solutions one at a time for sweetness. Panelists were instructed not to eat or drink (except water) for at least one hour before the test. During the test, panelists were instructed to sip each sample, swirl it around their mouth and then expectorate. After tasting each sample in the pair, panelists were instructed to record the sample that is “sweeter” in taste. Panelists cleansed their palates by rinsing with water, eating a cracker and waiting for an interval of about 5 minutes. Each pair was tasted twice. All samples were tasted at ambient temperatures. Compound 1 (at various concentrations) was evaluated in comparison to the control, 1.5% sucrose. The flavor and extract manufacturers association (FEMA) characterizes any compound that has a sweetness intensity greater than that of 1.5% in a water base as inherently sweet. FEMA recommends a 2-AFC test of the flavor modifier compared to that concentration (1.5% sucrose) to show that the flavor modifier does not have inherent sweetness.

Samples:

Control Sample (Sample 1): 1.5% sucrose in water

Test Samples (Sample 2):
10 μg/mL (10 ppm) Compound 1 in 0.2% ethanol in water
2 μg/mL (2 ppm) Compound 1 in 0.2% ethanol in water
0.5 μg/mL (500 ppb) Compound 1 in 0.2% ethanol in water

Eighteen subjects completed two replicates of a 2-AFC test for sweetness. Thirty three responses indicated the Control Sample was sweeter for each of the test samples evaluated. Three responses indicated the Test Sample was sweeter for each of the test samples evaluated. Using a beta binomial distribution, p=0.000 (two-sided alternative). Therefore, the Control Sample (1.5% sucrose) is significantly sweeter than all of the Test Samples evaluated (p<0.05). The results would indicate that concentrations of Compound 1 up to 10 μg/mL (10 ppm) do not elicit inherent sweetness.

TABLE 9
Inherent Sweetness of Compound 1

| | Sample 1 | | |
|---|---|---|---|
| | 1.5% sucrose | 1.5% sucrose | 1.5% sucrose |
| | Sample 2 | | |
| | Compound 1 | Compound 1 | Compound 1 |
| | @ 10 μg/mL | @ 2 μg/mL | @ 0.5 μg/mL |
| | in water | in water | in water |
| x (choosing Sample | 33 | 33 | 33 |
| 1 as sweeter) | | | |
| n | 36 | 36 | 36 |
| % correct | 92% | 92% | 92% |
| % needed | 67% | 67% | 67% |
| p-value | 0.000 | 0.000 | 0.000 |
| beta-binomial | 0.000 | 0.000 | 0.000 |
| Power | 100% | 100% | 100% |
| d′ | 1.956 | 1.956 | 1.956 |
| reverse p-value | 1.000 | 1.000 | 1.000 |

% Correct vs % Needed (95% Confidence, n = 18, reps = 2)

### Example 6: Evaluation of Compound 1 in a Mammalian Cell System Expressing a Bitter Taste Receptor

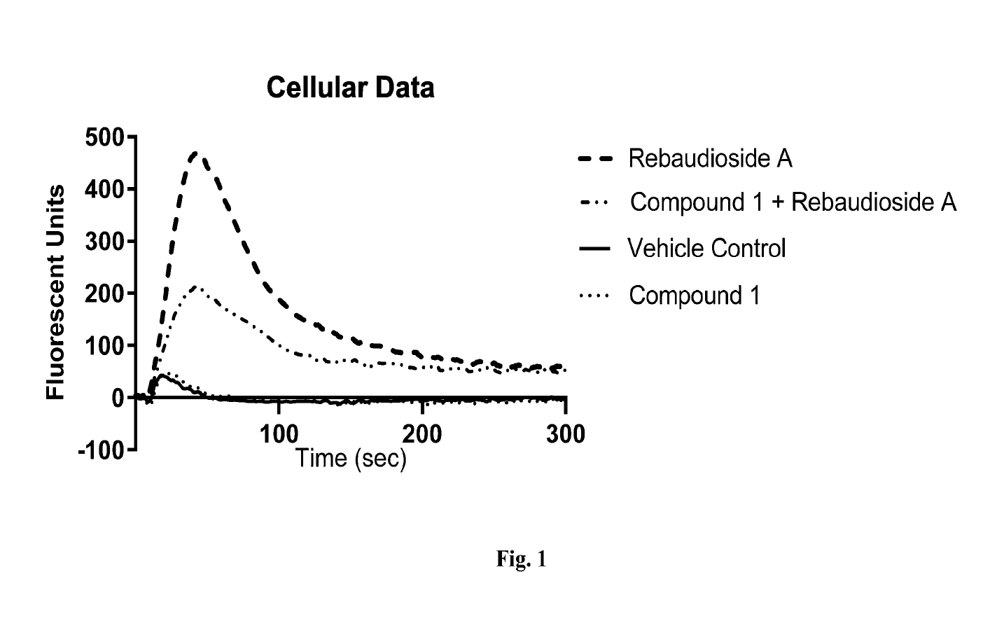

Compound 1 was evaluated for its effects on rebaudioside A in a mammalian cell system expressing a bitter taste receptor (T2R). Both the rebaudioside A and Compound 1 solutions were prepared in a standard HEPES buffer solution. Rebaudioside A was evaluated at a concentration of 825 μM and Compound 1 was evaluated at 3.2 mM (in the presence and absence of 825 μM rebaudioside A) and compared to vehicle control (HEPES buffer solution). Cells were incubated with a calcium responsive dye for one hour at 37 C and then exposed to rebaudioside A, Compound 1 (in the presence and absence of 825 μM rebaudioside A) and vehicle control. Change in fluorescence prior to compound addition and after compound addition was monitored for five minutes using a Hamamatsu Functional Drug Screening System (FDSS) 6000. The difference in fluorescence response over vehicle control was calculated using maximal cellular responses for each compound. Inhibition of the rebaudioside A response was observed with addition of Compound 1 as depicted in FIG. 1. The fluorescent response from Compound 1 on its own was not significantly different from vehicle control.

1. A composition comprising a compound according to Formula (I): 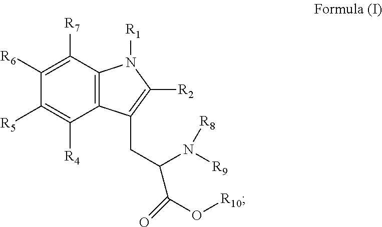
or a comestibly or biologically acceptable salt, derivative, diastereomer, or enantiomer thereof,
wherein, as valance and stability permit:
R₁ is independently H or C₁-C₆ alkyl;
R₂ is independently H or C₁-C₆ alkyl;
R₄ is independently H, OH, C₁-C₆ alkyl, or O(C₁-C₆) alkyl;
R₅ is independently H, OH, C₁-C₆ alkyl, or O(C₁-C₆) alkyl;
R₆ is independently H, OH, C₁-C₆ alkyl, or O(C₁-C₆) alkyl;
R₇ is independently H, OH, C₁-C₆ alkyl, or O(C₁-C₆) alkyl;
R₈ is independently H, C₁-C₆ alkyl, C(O)C₁-C₆ alkyl, C(O)R₁₁, or R₁₂;
R₉ is independently H, C₁-C₆ alkyl, C(O)C₁-C₆ alkyl, C(O)R₁₁, or R₁₂;
R₁₀ is independently H or C₁-C₆ alkyl; 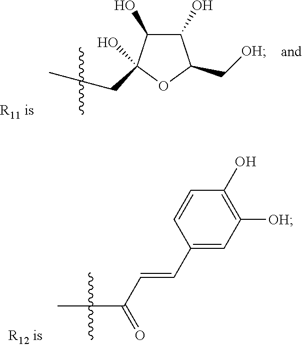 [SMILES: *C[C@]1(O)O[C@H](CO)[C@@H](O)[C@@H]1O ; *C(=O)/C=C/c1ccc(O)c(O)c1]
wherein the composition is edible and capable of reducing bitter taste of a bitter

2. The composition according to claim 1, or a comestibly or biologically acceptable salt, derivative, or enantiomer thereof, wherein as valence and stability permit:
R₁ is independently H or C₁-C₆ alkyl;
R₂ is independently H or C₁-C₆ alkyl
R₄ is independently H, OH, C₁-C₆ alkyl, or O(C₁-C₆) alkyl;
R₅ is independently H, OH, C₁-C₆ alkyl, or O(C₁-C₆) alkyl;
R₆ is independently H, OH, C₁-C₆ alkyl, or O(C₁-C₆) alkyl;
R₇ is independently H, OH, C₁-C₆ alkyl, or O(C₁-C₆) alkyl;
R₈ is independently H, C₁-C₆ alkyl, or C(O)C₁-C₆ alkyl;
R₉ is independently H, C₁-C₆ alkyl, or C(O)C₁-C₆ alkyl; and
R₁₀ is independently H or C₁-C₆ alkyl.

3. The composition according to claim 1, or a comestibly or biologically acceptable salt, derivative, or enantiomer thereof, wherein as valence and stability permit:
R₁ is H;
R₂ is H;
R₄ is independently H, OH, or O(C₁-C₆) alkyl;
R₅ is independently H, OH, or O(C₁-C₆) alkyl;
R₆ is independently H, OH, or O(C₁-C₆) alkyl;
R₇ is independently H, OH, or O(C₁-C₆) alkyl;
R₈ is independently H or C₁-C₆ alkyl;
R₉ is independently H or C₁-C₆ alkyl; and
R₁₀ is independently H or C₁-C₆ alkyl.

4. The composition according to claim 1, or a comestibly or biologically acceptable salt, derivative, or enantiomer thereof, wherein as valence and stability permit:
R₁ is H; R₂ is H; R₄ is H; R₅ is H; R₆ is H; R₇ is H; R₈ is H; R₉ is C₁-C₆ alkyl; and R₁₀ is H.

5. The composition according to claim 1, wherein said compound according to Formula (I) is: 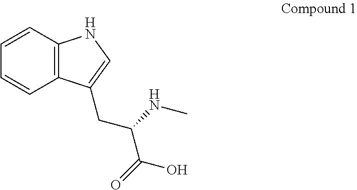 [SMILES: CN[C@@H](Cc1c[nH]c2ccccc12)C(=O)O]
or a comestibly or biologically acceptable salt, derivative, or enantiomer thereof.

6. The composition of any one of claims 1-5, wherein the composition further comprises a bitter tastant.

7. The composition according to claim 6, wherein the bitter tastant is a foodstuff.

8. The composition of according to claim 6, wherein the bitter tastant is a high potency sweetener, selected from stevioside or rebaudioside.

9-10. (canceled)

11. The composition of claim 9 wherein the composition further comprises sugar.

12. A food product comprising the composition of any one of claims 1-5.

13-14. (canceled)

15. A method of preparing an edible composition comprising:
(a) providing a comestibly acceptable carrier; and
(b) adding to the comestibly acceptable carrier a compound according to Formula (I), or a comestibly or biologically acceptable salt, derivative, diastereomer, or enantiomer thereof; or Compound 1, or a comestibly or biologically acceptable salt, derivative, or enantiomer thereof; or combinations of any of the aforementioned.

16. The method according to claim 15, wherein said comestibly acceptable carrier is inherently bitter.

17. The method according to claim 15 or 16, wherein the edible composition further comprises sugar.

18. The method according to claim 17, wherein the method further comprises:
(c) adding a bitter tastant.

19. The method according to claim 18, wherein the bitter tastant comprises a high potency sweetener.

20-21. (canceled)

22. A method of reducing the amount of sugar in an edible composition comprising:
(a) replacing an amount of sugar present an edible composition with an amount of a high potency sweetener; and
(b) incorporating into the edible composition an effective amount of a compound according to Formula (I), or a comestibly or biologically acceptable salt, derivative, diastereomer, or enantiomer thereof; Compound 1, or a comestibly or biologically acceptable salt, derivative, or enantiomer thereof; or combinations of any of the aforementioned.

23-26. (canceled)

27. The method according to claim 22, wherein the edible composition maintains a sweet flavor.

28. The method according to claim 27, wherein the high potency sweetener is stevioside or rebaudioside A.

29. (canceled)

30. A method of reducing bitter taste attributed to a bitter tastant in an edible composition comprising: adding an effective amount of a compound according to Formula (I), or a comestibly or biologically acceptable salt, derivative, diastereomer, or enantiomer thereof; Compound 1, or a comestibly or biologically acceptable salt, derivative, or enantiomer thereof; or combinations of any of the aforementioned, to the edible composition such that any bitter taste induced by the bitter tastant is reduced.

31. (canceled)

32. The method according to claim 30, wherein the edible composition is a food product, a consumer product, or a pharmaceutical composition.

33-52. (canceled)
